(19) United States
(12) Patent Application Publication
(10) Pub. No.: US 2003/0198973 A1
(43) Pub. Date: Oct. 23, 2003

(54) Chimeric fusion molecule for analyte detection and quantitation

(75) Inventors: Ian E. Burbulis, Berkeley, CA (US); Robert H. Carlson, Los Angeles, CA (US)

Correspondence Address: TRASK BRITT, P.O. BOX 2550, SALT LAKE CITY, UT, 84110, US

(73) Assignee: THE MOLECULAR SCIENCES INSTITUTE, INC.

(21) Appl. No.: 10/218,233

(22) Filed: Aug. 12, 2002

Related U.S. Application Data
(60) Provisional application No. 60/374,795, filed on Apr. 23, 2002.

(51) Int. Cl. C12Q 1/68; G01N 33/53; C12P 21/04; C12P 19/34; C12N 15/87

(52) U.S. Cl. 435/6; 435/7.1; 435/69.7; 435/320.1; 435/325; 435/455; 435/91.2

(57) ABSTRACT

Detector fusion molecules are produced by attaching a protein sub-unit to a linker, and attaching the linker to a nucleic acid molecule. The detector fusion molecules have utility in detecting and quantifying a specific target analyte from a sample. The protein sub-unit of the detector fusion molecule is selected to specifically bind the specific target analyte. The nucleic acid molecule of the detector fusion molecule is used as a tag, thus allowing for the detection and quantification of the target analyte. The sample is contacted with detector fusion molecules, thereby allowing detector fusion molecules to specifically bind any specific target analytes in the sample. The nucleic acid molecule of the detector fusion molecule is amplified using known processes, thereby producing an amplification product. The amplification product is detected and quantified, thus determining an amount of the target analyte in the sample.

### RELATED APPLICATIONS

This application is a continuation-in-part application of U.S. Provisional Patent Application Serial No. 60/374,795 that was filed on Apr. 23, 2002.

### BACKGROUND

1. The Field of the Invention

The invention disclosed herein relates generally to the assay of samples of molecules involved in a biological activity. More specifically, the present invention relates to detector molecules of the type used to detect and quantify target analytes in such samples. The present invention has particular applicability to the detection and quantitation of samples of human protein molecules.

2. Background Art

Protein molecules are produced by the cells of living organisms and are essential participants in most biological processes. Typically, each protein molecule in a living organism performs a specific function, such as facilitating a given metabolic activity or transporting chemical constituents. The specific function of a given protein molecule is determined by the sequence of amino acid building blocks that are connected in end-to-end relationship to make up the protein molecule. The precise sequence of the amino acids in each protein molecule is in turn a coded replication of a portion of the sequence of nucleotide building blocks in some gene in the genome of the organism in which the protein molecule has utility. A gene will thus embody a coded version of each protein molecule that corresponds thereto. Thus, the biological function of a protein molecule may be ascertained by studying the gene to which the protein molecule corresponds.

One process that can be used to determine the function of a protein molecule is the process of forward genetics. In forward genetics, the correlation of functions to protein molecules commences with the identification of a function occurring in an organism and proceeds to locate a protein that performs that function by making reference to the sequence of nucleotides in genes of the genome of the organism. The organism is subjected to conditions that cause the genome of the organism to change or mutate. Correspondingly, a mutant organism will result that is studied closely to identify functions present in the original organism that have been lost in the mutated organism. Correspondingly, the mutated genome is compared closely with the original genome to detect structural changes between the original genome and the mutated genome that can account for the observed loss of function between the original organism and the mutated organism. The protein encoded by any portion of the original genome not faithfully repeated in the mutated genome is then investigated as a candidate protein molecule that performs the function lost during the mutation process.

Although forward genetics can be used successfully to correlate functions to protein molecules in lower organisms, such as bacterial forward genetics is inappropriate for correlating function to protein molecules in humans, because humans cannot ethically be randomly mutated.

Instead, the contrasting process of reverse genetics is used to identify the functions of protein molecules in higher organisms. Reverse genetics is conducted in conjunction with proteomics, a study of protein molecules produced by cells in which the function of protein molecules is established by isolating and studying the protein molecules.

Reverse genetics commences with the known sequences of genes in the genome of a higher organism. Using known nucleotide sequence information for an individual gene, a protein encoded by the gene is produced. Then proteomics is used to determine the function of that protein molecule.

Although the function of individual protein molecules is determinable using reverse genetics, there are at least 30,000 genes in a human cell, and collectively these genes are estimated to be capable of producing between 300,000 and one million different proteins. One obstacle to using reverse genetics to rapidly establish the function of each protein molecule in a human or other higher organism, is that the determination of the function of a single human protein using proteomics yet requires substantial time.

Aberrant or mutant forms of protein molecules disrupt normal biological processes causing disease, including some cancers and inherited disorders, such as cystic fibrosis and hemophilia. Given sufficient time, it is hoped that the functions of the protein molecules produced by the cells of a healthy person can be established. Then any aberrant or mutant protein molecules not normally present in the cells of a healthy person can be detected. Abnormal protein molecules can then be used as markers indicating that cells are in a disease state.

One way that disease markers can be detected is by developing detector molecules that specifically bind, or attach, to given disease markers. To diagnose a patient for a disease, the blood of the patient is tested with a detector molecule corresponding to that disease. If the detector molecule does bind, the existence of the disease marker becomes apparent, and medical personnel can conclude that the specific disease to which the disease marker corresponds is present in the patient.

The identification of a disease marker associated with a given disease can yield new products that prevent, diagnose, or treat the corresponding disease. For example, detector molecules can be used to isolate given disease markers. Then the disease markers may be studied using proteomics.

One problem in the diagnosis of diseases in this manner is that some human proteins have not been characterized, and some diseases are as yet not diagnosable. Another drawback in diagnosing diseases in this manner is that some diseases produce only small numbers of disease markers in the blood of a victim, and thus cannot be visualized using known methods. Although detector molecules will bind to whatever corresponding disease markers are present in the blood, if the number of disease markers in a blood sample is few, known processes may not be sufficiently sensitive to permit those disease markers to even be detected.

Infections are caused by pathogenic microorganisms that invade the body of a patient. The pathogenic microorganisms produce virulence proteins, such as toxins, that then damage the tissues of the patient. Virulence proteins are, however, detectable in blood. Thus, the blood of a patient can be used to determine whether the patient is infected with a pathogenic microorganism. Detector molecules corresponding to specific virulence proteins are added to a blood sample. If detector molecules bind to a constituent of the sample, one or more of those specific virulence proteins are known to be in the sample, and the presence in the patient of an infection that produce virulence proteins is confirmed.

Since all virulence proteins of pathogenic microorganisms have not been identified, some infections are not diagnosable in this manner. Other infections are not diagnosable until late in the course of an infection, because the number of virulence proteins produced by the pathogenic microorganism early in the course of the infection is too small to be identified in the blood sample.

Two primary types of detector molecules are used to bind target analytes: antibodies and fusion molecules. Each type of detector molecule will be discussed individually. The term “target analyte” will be used herein to refer to the molecule that becomes bound by a given detector molecule. Examples of target analytes are disease markers, virulence proteins, nucleic acid molecules, protein sub-units, sugars, and lipids.

A first type of detector molecule used to bind target analytes is an antibody detector molecule. The antibody portion of the antibody detector molecule is a protein produced by the immune system of an animal in response to a target analyte foreign to the animal. Antibodies that bind specifically to the target analyte are generated by immunizing an animal with the target analyte itself. These antibodies bind to a specific site, or epitope, on the target analyte that was used to immunize the animal.

Antibodies produced by an animal in response to a target analyte are collected from the animal, and the antibodies are tagged with a detectable marker to form an antibody detector molecule.

Typically, the detectable marker is a chemical moiety that emits fluorescence, emits radioactivity, or exhibits enzymatic activity. The antibody detector molecule binds specifically to the target analytes that caused the antibody detector molecule to be produced. To determine whether the antibody detector molecule is bound to the target analyte, the presence of the detectable marker is sensed by searching for the fluorescence, the radioactivity, or the enzymatic activity that is reflective of the presence of the detectable marker by a given of the antibody detector molecule.

Although antibodies bind with a high specificity to the target analyte that caused the antibody to be produced, the immunization of an animal to generate antibodies, and the subsequent collection of the antibodies from the animal can take months to accomplish, representing a problem when the antibody detector molecules are needed in short order. Also, antibodies cannot be produced for some target analytes, because some target analytes do not generate an immune response in an animal.

Another disadvantage in using antibody detector molecules is that the detectable markers used to tag the antibodies are not capable of being amplified, or readily reproduced in a large number. Therefore, when a small number of antibody detector molecules bind to target analytes within a sample, the correspondingly small number of detectable markers in the sample cannot be detected, because the signal emitted by these correspondingly small number of detectable markers is too weak to be sensed by known processes. For instance, the sensor used to detect the emitted signal from the fluorescence, the radioactivity, or the enzymatic activity may be present, but may not be sensitive enough to detect the weak signal.

Although the presence of target analytes in a sample can be detected using antibody detector molecules, the number of target analytes in a sample can only be estimated based on the relative amount of signal emitted from the detectable markers. The number of target analytes in the sample cannot be precisely counted, because the detector molecules of the antibody detector molecules are not capable of being amplified in a linear fashion.

A second type of detector molecule used to detect target analytes is a fusion molecule. A fusion molecule has a protein sub-unit linked to a ribonucleic acid molecule by a covalent bond. The protein sub-unit portion of the fusion molecule binds to the target analyte, and the ribonucleic acid molecule is used to announce the presence in a sample of a fusion molecule bound to that target analyte.

Ribonucleic acid molecules are not particularly stable. The environment within a cell contains numerous enzymes that degrade ribonucleic acid molecules. Therefore, in a study of proteins in a cell, enzymes from the environment of the cell degrade the ribonucleic acid portion of a fusion molecule of this type. Fusion molecules made up of a protein sub-unit linked to a ribonucleic acid molecule are thus not well suited for the study of proteins in living cells.

To studying a given protein molecule itself to ascertain the function of the given protein molecules, the function of the given protein molecules may be determined by identifying a target analyte that interacts with the given protein molecules. For example, a given protein molecule may bind to a deoxyribonucleic acid molecule of a gene in order to regulate expression of the gene. The function of the given protein molecule may thus be determined by identifying the gene that interacts with the given protein molecule.

The target analyte interacting with a given protein molecule in a cell may be determined by disrupting the cell to release the contents of the cell, including the given protein molecule and the target analyte. The contents of the cell are so treated as to link the given protein molecule to the target analyte, forming a complex. A detector molecule specific for the given protein molecule or the target analyte is used to isolate the complex. The target analyte in the complex is identified; and based on the identity of the target analyte, the function of the protein is inferred.

A complication exists in ascertaining the function of a given protein molecule in this manner, if the given protein molecule interacts non-specifically with multiple target analytes. If the given protein molecule non-specifically interacts with a random target analyte, the precise function of the given protein molecule will be improperly determined if the random target analyte bound thereto. Also, if the given protein does not interact with any target analyte in a cell, the biological function of the protein cannot be determined in this manner.

### SUMMARY OF THE INVENTION

It is thus a broad object of the present invention to improve the processes used to study human proteins, thereby to improve molecular research and human healthcare.

It is also an object of the present invention to increase the speed and efficiency with which the function of human protein molecules can be determined.

It is a further object of the present invention to characterize unknown disease markers associated with diseases in humans.

An additional object of the present invention is to increase the sensitivity of detector molecules used to identify disease markers. A related object of the present invention is to identify unknown virulence proteins produced by pathogenic microorganisms.

Another object of the present invention is to decrease the amount of time required to produce detector molecules.

Yet another object of the present invention is to produce a detector molecule for target analytes that does not produce an immune response in an animal.

An additional object of the present invention is a detector molecule with an amplifiable detectable marker for more efficient detection that can be accurately quantified.

A further object of the present invention is an improved process for identifying a target analyte that interacts with a given protein molecule.

The present invention also has as an object a detector molecule that minimizes cross-linking and non-specific interactions with more than one target analyte.

Yet another object of the present invention is a detector fusion molecule that is stable for use in many environments. A related object of the present invention is thus a detector fusion molecule that will not be degraded by the environment of a cell.

To achieve the foregoing objects, and in accordance with the invention as embodied and broadly described herein, systems and methods are described for producing detector fusion molecules. Also provided are detector fusion molecules having detectable markers attached thereto.

In one aspect of the present invention, a method is provided incorporating teachings of the present invention that produces a fusion molecule useable as a detector fusion molecule for a predetermined target analyte. The method includes the step of attaching a reactive moiety to a first end of a protein sub-unit, thereby creating a reactive intermediate with the reactive moiety attached at a first end thereof. A coupling reagent is bonded to a first end of a nucleic acid molecule, forming a modified nucleic acid molecule. A reaction is catalyzed between the reactive moiety of the reactive intermediate and the coupling reagent of the modified nucleic acid molecule. The reaction displaces the reactive moiety from the first end of the reactive intermediate and forms a covalent bond between the first end of the reactive intermediate and the first end of the modified nucleic acid molecule.

One example of a coupling reagent that may be bonded to the first end of the nucleic acid molecule to form a modified nucleic acid molecule useful in the inventive method is a phosphoramidite-containing molecule. In another example, a cysteine-like moiety is attached to a nucleotide, thereby forming a modified nucleotide. The modified nucleotide is then linked to the first end of a nucleic acid molecule, thereby forming a modified nucleic acid molecule.

Examples of protein sub-units useful in the methods of the present invention include natural proteins, recombinant peptide aptamers, and synthetic peptides. Examples of nucleic acid molecules useful in the present invention comprise deoxyribonucleic acid molecules, double-standard deoxyribonucleic acid molecules, ribonucleic acid molecules, and peptide nucleic acid

In another aspect of the present invention, a fusion molecule is described that binds to a predetermined target analyte. The fusion molecule includes a protein sub-unit, a linker attached to a first end of the protein sub-unit, and a deoxyribonucleic acid molecule attached at a first end thereof to the linker by a covalent bond.

Examples of linkers used in a fusion molecule incorporating teachings of the present invention include an amide linkage or a cysteine-like moiety attached to the carboxyl terminus of the protein sub-unit. The linker is covalently bonded to the 5′ end or the 3′ end of the deoxyribonucleic molecule by an amide bond.

In yet another aspect of the present invention, a detector fusion molecule comprises a protein sub-unit, a cysteine-like moiety attached to a first of the protein sub-unit, and a nucleic acid molecule attached at a first end thereof to the cysteine-like moiety by a covalent bond.

In one example, the cysteine-like moiety of the protein sub-unit comprises an amide linkage. Examples of nucleic acid molecules of the detector fusion molecule include deoxyribonucleic acid molecules, ribonucleic acid molecules, double-stranded deoxyribonucleic molecules, and peptide nucleic acid molecules.

Another aspect of the present invention includes a method for recognizing a target analyte in a sample. The method includes the step of manufacturing detector fusion molecules by attaching a quantity of reactive moieties to a first end of a quantity of protein sub-units, thereby creating a quantity of reactive intermediates with reactive moieties attached at a first end thereof. A quantity of coupling reagents is bonded to first ends of a quantity of nucleic acid molecules, thereby forming a quantity of modified nucleic acid molecules. A reaction is catalyzed between the reactive moieties of the reactive intermediates and the coupling reagents of the modified nucleic acid molecules. As a result of the reaction, the reactive moieties are displaced from the first end of the reactive intermediates, and covalent bonds are formed between the first end of the reactive intermediates and the first end of the modified nucleic acid molecules. The sample is contacted with a quantity of the detector fusion molecules, and the detector fusion molecules bind to target analytes in the sample. The nucleic acid molecules of the detector fusion molecules bound to the target analyte are then amplified, producing an amplification product. The amount of target analyte in the sample is quantified by determining the amount of the amplification product.

In one embodiment of the present invention, the nucleic acid molecule amplification is accomplished by hybridizing to the modified nucleic acid molecule of the bound detector fusion molecule a primer having a detectable marker and a nucleotide sequence complementary to a portion of a sequence of the modified nucleic acid molecule. A deoxyribonucleic acid polymerase is added to the bound detector fusion molecules. After amplification, the amount of the detectable marker is measured, thereby quantifying the amount of target analyte present in the sample.

In a further aspect of the present invention, a kit for recognizing or quantifying a target analyte is provided. The kit includes a detector fusion molecule comprising a protein sub-unit, a linker attached to a first end of the protein sub-unit, and a deoxyribonucleic molecule attached at a first end thereof to the linker by a covalent bond. The kit also includes a first means for amplifying the detector fusion molecule to produce an amplification product and a second means for visualizing the amplification product.

An example of such a first means is a deoxyribonucleic acid primer and a deoxyribonucleic acid polymerase. An example of such a second means is a detectable marker attached to the deoxyribonucleic acid primer.

Additional objects and advantages of the invention will be set forth in the description which follows and, in part, will be obvious from the description or may be learned by the practice of the invention. The objects and advantages of the invention may be realized and obtained by means of the instruments and combinations pointed out in the appended claims.

### BRIEF DESCRIPTION OF THE DRAWINGS

In order that the manner in which the above-recited and other advantages and objects of the invention are obtained, amore particular description of the invention briefly described above will be rendered by reference to a specific embodiment thereof which is illustrated in the appended drawings. Understanding that these drawings depict only a typical embodiment of the invention and are not, therefore, to be considered limiting of its scope, the invention will be described and explained with additional specificity and detail through the use of the accompanying drawings in which:

FIG. 1 is a perspective view of a clinical setting in which a medical practitioner is taking a sample of blood from a patient for analysis;

FIG. 2 is a schematic representation of a detector fusion molecule produced incorporating teachings of the present invention;

FIG. 3 is a schematic representation of the detector fusion molecule of FIG. 2 bound to a target analyte, wherein a portion of the detector fusion molecule has been amplified using methods incorporating teachings of the present invention;

FIG. 4 is a schematic representation of three detector fusion molecules illustrating nucleic acid portions with varying lengths produced incorporating teachings of the present invention;

FIG. 5 is a schematic representation of steps in a first embodiment of a method incorporating teachings of the present invention for producing a first embodiment of a detector fusion molecule that includes RNA;

FIG. 6 is a flowchart depicting generally the production and use of detector fusion molecules produced using steps in a second embodiment of a method embodying teachings of the present invention;

FIG. 7 a schematic representation of steps in a method for making a reactive intermediate having utility in producing a detector fusion molecule incorporating teachings of the present invention;

FIG. 8 is a photograph of a first sample of a protein sub-unit-intein recombinant protein as shown in FIG. 7 on an acrylamide gel and stained with Coomassie Blue;

FIGS. 9A, 9B and 9C are schematic representations of steps in a first embodiment of a method for using a phosphoramidite-containing moiety to convert a nucleic acid into a first embodiment of a modified nucleic acid having utility in producing a detector fusion molecule incorporating teachings of the present invention;

FIG. 10 is a photograph of a first sample of a modified nucleic acid molecule having utility in producing a detector fusion molecule incorporating teachings of the present invention run on an agarose gel and stained with ethidium bromide;

FIG. 11 is a schematic representation of a second embodiment of a method used to convert a nucleic acid into a second embodiment of a modified nucleic acid having utility in producing a detector fusion molecule incorporating teachings of the present invention;

FIG. 12 is a schematic representation of steps in a first embodiment of a method for producing a first embodiment of a modified nucleotide having a cysteine-modified nucleotide and being useful to produce a detector fusion molecule incorporating teachings of the present invention;

FIG. 13 is a diagram of the molecular structure of a second modified nucleotide used to produce a detector fusion molecule incorporating teachings of the present invention;

FIG. 14 is a diagram of the molecular structure of a third modified nucleotide used to produce a detector fusion molecule incorporating teachings of the present invention;

FIG. 15 is a schematic representation of steps in a first embodiment of a method for using the modified nucleotide of FIG. 12 to convert a nucleic acid into a third embodiment of a modified nucleic acid having utility in producing a detector fusion molecule incorporating teachings of the present invention;

FIG. 16 is a schematic representation of steps in a method for producing a detector fusion molecule embodying the teachings of the present invention;

FIG. 17 is a schematic representation of steps in a second embodiment of a method incorporating teachings of the present invention for binding a target analyte and amplifying the detector fusion molecule bound to the target analyte;

FIG. 18 is a photograph of a first sample of an amplification products;

FIG. 19 is a photograph of a second sample like FIG. 18 of amplification products generated using T7 polymerase from detector fusion molecules designed to bind biotin incorporating teachings of the present invention run out on an acrylamide gel and stained with

FIG. 20A is a schematic representation in partial perspective of the processing with magnetic microparticles of a sample containing target analytes immobilized on a substrate;

FIG. 20B is a schematic representation of steps in a method for binding detector fusion molecules incorporating teachings of the present invention to target analytes on a single of the magnetic microparticles of FIG. 20A;

FIG. 20C is a schematic representation of steps in a second embodiment of a method incorporating teachings of the present invention for amplifying and detecting or quantifying, for example, the detector fusion molecules bound to target analytes on the magnetic microparticle of FIG. 20B;

FIG. 21 is a schematic representation of steps in a third embodiment of a method incorporating teachings of the present invention for amplifying and detecting or quantifying the detector fusion molecules of the present invention;

FIG. 22A is a photograph of an acrylamide gel stained with Coomassie Blue depicting a first target analyte used to produce detector fusion molecules incorporating teachings of the present invention;

FIG. 22B is a photograph of a third sample of amplification products generated using T7 polymerase from detector fusion molecules designed to bind the target analyte of FIG. 22A incorporating teachings of the present invention run out on an acrylamide gel and stained with Sybrgreen 2;

FIG. 23 is a graph of the fluorescence of the amplification products of the sample of detector fusion molecules of FIG. 22B relative to corresponding concentrations of the target analyte;

FIG. 24A is a schematic representation of a detector fusion molecule embodying teachings of the present invention and having utility in building a nanostructure;

FIG. 24B is a schematic representation of a nanostructure manufactured using the detector fusion molecule of FIG. 24A; and

FIG. 25 is a perspective view of a kit containing reagents used to amplify or quantitate detector fusion molecules produced embodying the teachings of the present invention.

### DESCRIPTION OF THE PREFERRED EMBODIMENT

The following definitions are provided at the outset to facilitate the following descriptions. As used herein, the term “epitope” will be used to refer to a specific area, portion, or domain of a target analyte that associates, or binds, to a detector fusion molecule. The expression “mRNA” as used herein will be used to refer to messenger ribonucleic acid. As used herein, the expression “dNTP” will be used to refer to deoxynucleotide triphosphate. The expression “rNTPs” will be used herein to refer to ribonucleotide triphosphates. As used herein, the expression “DNA” will be used to refer to deoxyribonucleic acid, and the expression “RNA” will be used to refer to ribonucleic acid. The expression “PCR” will be used herein to refer to a polymerase chain reaction. As used herein, the expression “dUTP” will be used to refer to deoxyuracil triphosphate, the expression “dATP” will be used to refer to deoxyadenine triphosphate; the expression “dCTP” will be used to refer to deoxycytidine triphosphate; and the expression “dGTP” will be used to refer to deoxyguanosine triphosphate.

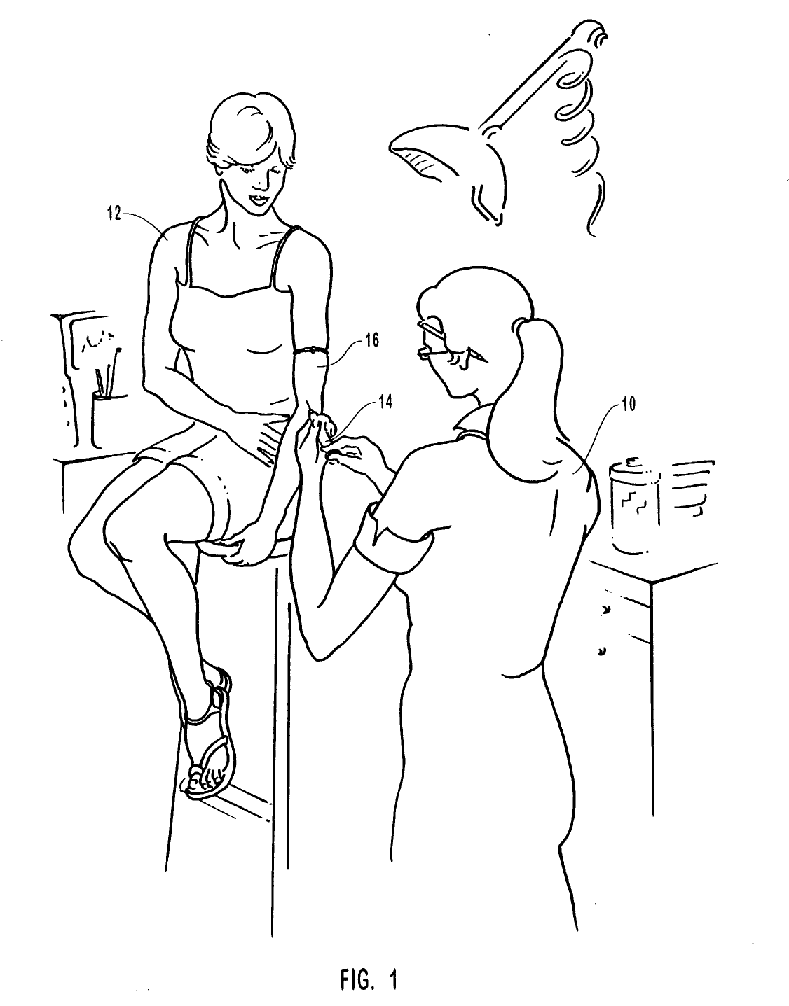

FIG. 1 depicts a clinical setting in which a medical practitioner 10 is obtaining a blood sample from an arm 16 of a patient 12 using a syringe 14. According to one aspect of the teachings of the present invention, the blood sample from patient 12 is treated with a detector fusion molecule or a plurality of detector fusion molecules produced using the teachings of the present invention. Such detector fusion molecules are designed to specifically bind to a disease marker representative of a particular disease in patient 12. Alternatively, the detector fusion molecules are designed to specifically bind a virulence protein indicative of the infection of patient 12 with a specific microorganism. Detector fusion molecules that bind disease markers or virulence proteins present in the blood sample from patient 12 are introduced into the blood sample from patient 12. If the detector fusion molecules bind to such target analytes in the blood sample from patient 12, the detector fusion molecules are detected and quantified using methods described herein, and medical practitioner 10 diagnoses an illness in patient 12.

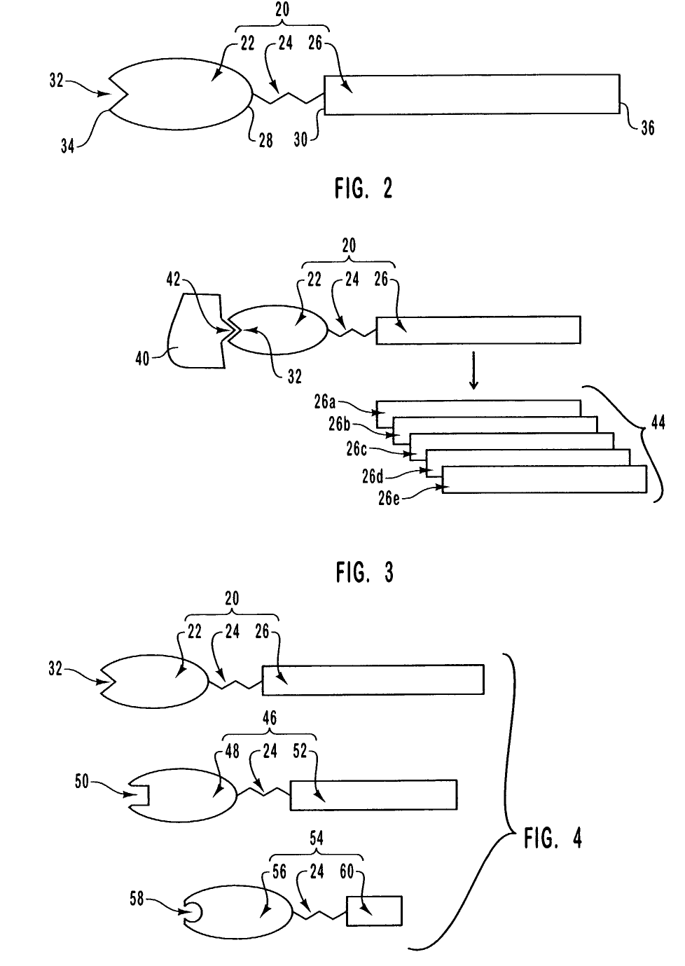

FIG. 2 illustrates a detector fusion molecule 20 produced according to methods incorporating teachings of the present invention which has utility in testing the blood sample of patient 12 of FIG. 1. Detector fusion molecule 20 includes a linker 24 interconnecting a protein sub-unit 22 and a nucleic acid molecule 26. Although protein sub-units 22 may be complex three-dimensional structures, for ease of illustration, protein sub-unit 22 is illustrated in FIG. 2 as an oval-shaped structure with a first end 28 and a second end 34. First end 28 of protein sub-unit 22 is attached to linker 24 by a covalent bond, and first end 28 of protein sub-unit 22 is the carboxyl terminus of protein sub-unit 22. Second end 34 of protein sub-unit 22 includes a binding site 32. Linker 24 is attached to a first end 30 of nucleic acid molecule 26 by a covalent bond. A second end 36 of nucleic acid molecule 26 is shown opposite first end 30 of nucleic acid molecule 26.

Protein sub-unit 22 in the generalized embodiment depicted in FIG. 2 is a polypeptide chain. Polypeptide chains include various amino acid building blocks selected from a group of about twenty amino acids. These amino acids are connected chemically with peptide bonds to form a chain of amino acids referred to as a polypeptide chain. Protein sub-unit 22 may be derived from one of three sources and may be any polypeptide that binds target analyte very specifically. First, protein sub-unit 22 may comprise a natural protein normally found in an organism. The natural protein may be isolated from the organism using known processes. Second, protein sub-unit 22 may comprise a recombinant peptide aptamer or any other recombinant protein. Recombinant peptide aptamers are produced using known recombinant protein technologies, such as cloning a gene into a vector. One type of recombinant peptide aptamer is a recombinant antibody, such as a single chain fragment variable of an antibody. The cloned gene is expressed in a cell, and the resultant translated peptide aptamer is purified using known processes. Third, protein sub-unit 22 may be a synthetic peptide produced in vitro using known processes for chemically synthesizing polypeptide chains.

Linker 24 of detector fusion molecule 20 of FIG. 2 may comprise an amide linkage or a cysteine-like moiety. Nucleic acid molecule 26 of FIG. 2 may comprise any type of nucleic acid molecule. For instance, nucleic acid molecule 26 may be ribonucleic acid, deoxyribonucleic acid, double-stranded deoxyribonucleic acid, peptide nucleic acid, or a hybrid nucleic acid molecule comprising more than one type of nucleic acid molecule. As shown, nucleic acid molecule 26 is attached to linker 24 on first end 30 of nucleic acid molecule 26. Since all nucleic acid molecules have a 5′ and a 3′ end, and as all nucleic acid molecules form complex three-dimensional structures, the depiction of nucleic acid molecule 26 with first end 30 and second end 36 is for ease of explanation. First end 30 of nucleic acid molecule 26 is attached to linker 24 on the 5′ end of nucleic acid molecule 26. In alternative embodiments, linker 24 maybe attached to nucleic acid molecule 26 on the 3′ or the second end 36 thereof

Detector fusion molecule 20 of FIG. 2 is depicted in FIG. 3 bound to a target analyte 40. The binding site 32 of protein sub-unit 22 interacts with an epitope 42 of the target analyte 40. The binding of epitope 42 to binding site 32 is due to molecular associations including, without limitation, hydrogen bonds, hydrophobic forces, hydrophilic forces, and ionic bonds between epitope 42 of target analyte 40 and binding site 32 of protein sub-unit 22. Due to the various types of molecular associations that may be present in binding protein sub-unit 22 to target analyte 40, protein sub-units 22 may be selected to bind any chemical compound including, without limitation, other proteins, polypeptides, sugars, carbohydrates, lipids, metals, polysacharrides, nucleic acids, minerals, and metabolites.

FIG. 3 illustrates an amplification product 44 produced by amplifying nucleic acid molecule 26 of detector fusion molecule 20. As illustrated, amplification product 44 includes a plurality of nucleic acid molecules 26a through 26e that have been amplified from nucleic acid molecule 26 of detector fusion molecule 20. The plurality of nucleic acid molecules 26a through 26e of amplification product 44 are substantially identical in size and comprise essentially the same nucleotide sequence as nucleic acid molecule 26 of detector fusion molecule 20.

Referring now to FIG. 4, there is shown a schematic representation of detector fusion molecule 20 of FIG. 2 with distinct detector fusion molecule 46 and distinct detector fusion molecule 54.

Detector fusion molecule 46 includes a protein sub-unit 48 that is different from protein sub-unit 22 of detector fusion molecule 20. Protein sub-unit 48 has a binding site 50 that binds to an epitope such as, but possibly different from epitope 42 of target analyte 40 depicted in FIG. 2. Detector fusion molecule 46 also includes a nucleic acid molecule 52 that is shorter in length than nucleic acid molecule 26 of detector fusion molecule 20.

Detector fusion molecule 54 includes a protein sub-unit 56 different from protein sub-unit 48 of detector fusion molecule 46 and different from protein sub-unit 22 of detector fusion molecule 20. Detector fusion molecule 54 includes nucleic acid molecule 60 that is shorter in length than nucleic acid molecules 26 and 52, and a binding site 58 specific for an epitope different from that of detector fusion molecules 20 and 46.

The three different detector fusion molecules 20, 46, and 54 illustrated in FIG. 4 have different respective binding sites 32, 50, and 58. Therefore, detector fusion molecules 20, 46, and 54 bind respectively to different target analytes. Each detector fusion molecule produced using the teachings of the present invention may be designed with a different protein sub-unit, such that the various detector fusion molecules maybe used to bind a wide variety of target analytes. Detector fusion molecules 20, 46, and 54 include nucleic acid molecules 26, 52, and 60 of different length. As a result, the amplification products generated from each of detector fusion molecule 20, 46, and 54 have different lengths. Accordingly, the amplification products of detector fusion molecules 20, 46, and 54 are distinguishable on the basis of length when resolved on a gel.

Alternatively, nucleic acid molecules 26,52, and 60 may have different nucleotide sequences. The amplification products produced from the nucleic acid molecules 26, 52, and 60 are distinguishable on the basis of sequence. In this manner, each nucleic acid molecule 26, 52, and 60 of the three detector fusion molecules 20, 46 and 54 serves as a barcode identifying each detector fusion molecule 20, 46, and 54. For example, a sequence of nucleic acid molecule 26 may be the sequence encoding protein sub-unit 22. Accordingly, detector fusion molecules 20, 46, and 54 may be used to identify a corresponding number of separate target analytes present simultaneously in a single sample.

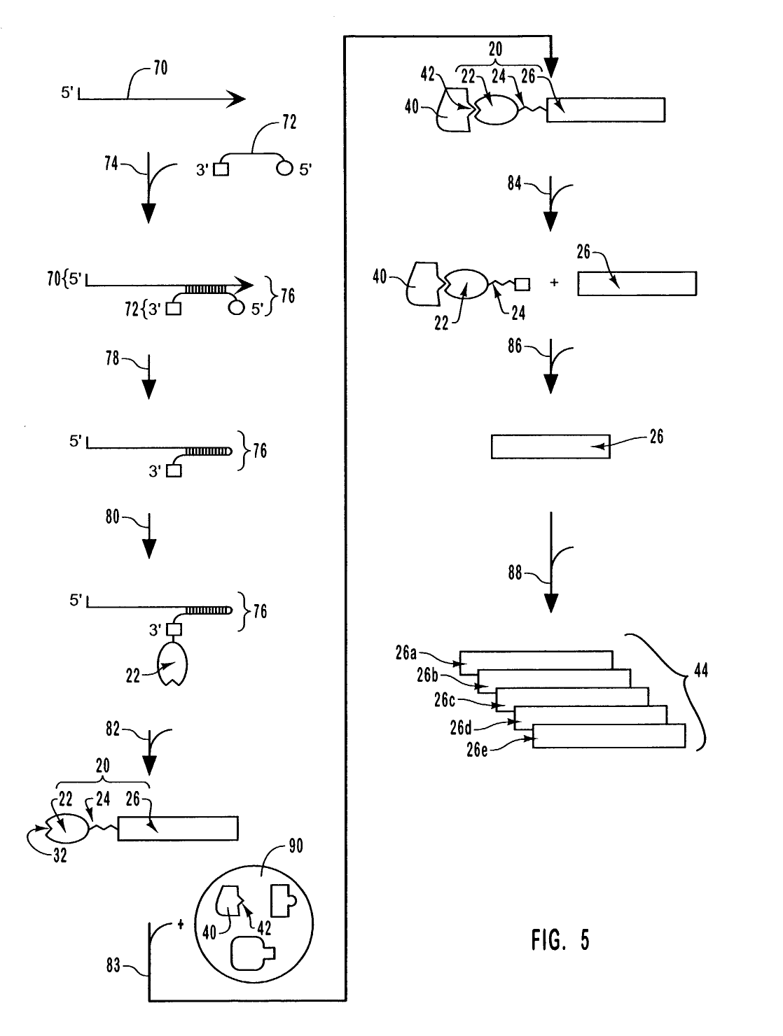

FIG. 5 depicts steps by which a detector fusion molecule may be produced using a first embodiment of a method embodying teachings of the present invention. As illustrated, commencing on the upper left of FIG. 5, a protein sub-unit encoding mRNA 70 is depicted as a linear strand. Protein sub-unit encoding mRNA 70 includes a ribonucleotide sequence that is complementary, at least in part, to a nucleotide sequence of a 3′-puromycin-5′-psoralin oligonucleotide 72. Protein sub-unit encoding mRNA 70 is hybridized to the 3′-puromycin-5′psoralin oligonucleotide 72 in a step 74 to form a hybridized complex 76. In a step 78, protein sub-unit encoding mRNA 70 portion is cross-linked to 3′-puromycin-5′-psoralin oligonucleotide portion 72 of hybridized complex 76, thereby covalently binding protein sub-unit encoding mRNA 70 to the 3′-puromycin-5′-psoralin oligonucleotide 72. In a step 80, hybridized complex 76 is translated in vitro, thus expressing protein sub-unit encoding mRNA 70 into a protein sub-unit 22 that remains attached to hybridized complex 76.

In a step 82, reverse transcriptase and dNTPs are added to a solution containing protein sub-unit 22 attached to hybridized complex 76, thus reverse transcribing the DNA of hybridized complex 76 to RNA and forming a mature detector fusion molecule 20. Nucleic acid molecule 26 of the detector fusion molecule 20 of FIG. 5 comprises a double-stranded nucleic acid molecule including one strand of RNA hybridized to a complementary strand of DNA.

Detector fusion molecule 20 is placed in contact with a sample 90 of target analytes at a step 83. Target analyte 40 with epitope 42 binds binding site 32 of detector fusion molecule 20. In a step 84, RNAse, an enzyme that degrades RNA is added to the solution containing detector fusion molecule 20 bound to target analyte 40. The RNAse degrades the ribonucleic acid molecule portion of nucleic acid molecule 26 and releases the DNA strand of nucleic acid molecule 26. Protein sub-unit 22 remains bound to target analyte 40 and to linker 24, which are separated from nucleic acid molecule 26. At a step 86, Klenow fragment, a DNA primer, and dNTPs are added to nucleic acid molecule 26, thereby allowing the Klenow fragment to polymerize the primer hybridized to the single-stranded DNA molecule and to form a double-stranded DNA molecule. At a step 88, T7 ribonucleic acid polymerase and rNTPs are added to produce amplification product 44.

Alternatively, at step a 88, Tax polymerase, primers complementary to each of the strands of the double-stranded DNA molecules, and dNTPs are added to the double-stranded DNA molecule, thereby producing an amplification product 44 that includes a plurality of nucleic acid molecules 26a through 26e. Whether amplification product 44 includes DNA or RNA as nucleic acid molecules 26a through 26e, amplification product 44 is detected using known processes to ascertain the presence of target analyte 40 in sample 90.

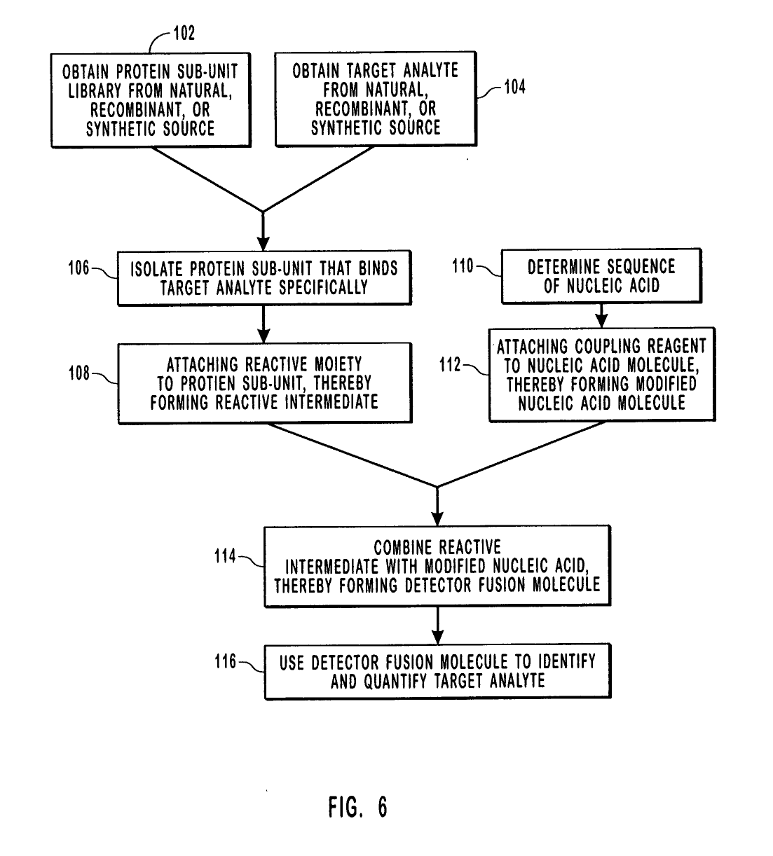

In FIG. 6 is shown a flowchart depicting generally the production and use of detector fusion molecules produced using steps in a second embodiment of a method embodying teachings of the present invention. As indicated in dialog boxes 102 and 104, the first steps of the illustrated method include obtaining a protein sub-unit library from a natural, recombinant, or synthetic source, or obtaining a target analyte from a natural, recombinant, or synthetic source.

A protein sub-unit library may be obtained at dialog box 102 from a natural source by isolating a plurality of naturally occurring genes and expressing the naturally occurring genes in an expression vector, thereby producing a library of natural protein sub-units. The expressed natural protein sub-units are proteins that have not been genetically modified or mutated, but are obtained from genes in a wild type state and isolated from an organism.

Alternatively, the protein sub-unit library could be obtained at dialog box 102 from a recombinant source where genes encoding the protein sub-units are genetically modified, such as by fusing the gene, or portion of a gene, to another gene, thereby producing a recombinant gene. The recombinant gene is expressed in an expression vector, thereby producing a library of recombinant protein sub-units.

The protein sub-unit library may also be obtained at dialog box 102 from a synthetic source, wherein genes encoding the protein sub-unit are randomly synthesized, thereby producing a synthetic gene. The synthetic gene is expressed in an expression vector, thereby producing a library of synthetic protein sub-units. Alternatively, the synthetic protein sub-unit may be randomly synthesized using a protein synthesizer.

A target analyte is obtained at dialog box 104 from a natural, recombinant, or synthetic source. The target analyte may comprise any type of molecule, including without limitation, nucleic acid molecules, polypeptide molecules, protein molecules, polysacharrides, lipids, metals, minerals, vitamins, or any other type of known molecule. In a manner similar to obtaining the protein sub-unit library at dialog box 102, the target analyte may be obtained from a natural source, such as a target analyte that is isolated in an unmodified form, such that the natural target analyte represents the target analyte in a wild-type state.

Alternatively, the target analyte may be obtained at dialog box 104 from a recombinant source. For instance, the recombinant target analyte may be produced by fusing one natural target analyte to another target analyte, or the recombinant target analyte could be produced from or comprise a recombinant protein. The target analyte may also be obtained at dialog box 104 from a synthetic source. For example, the target analyte may be synthetically manufactured in vitro.

Once the protein sub-unit library and the target analyte are obtained at dialog boxes 102 or 104, a protein sub-unit that specifically binds the target analyte is isolated using known processes at dialog box 106, such as phage display, yeast two-hybrid, yeast display, bacterial display, bacterial two-hybrid, surface plasmon resonance, or any technique that allows for the specific interaction of two molecules to be determined. Once the protein sub-unit that specifically binds the target analyte is isolated at dialog box 106, the gene encoding for the protein sub-unit is isolated, and as illustrated in dialog box 108, the isolated protein sub-unit is attached to a reactive moiety, thereby producing a reactive intermediate.

Referring to dialog box 110, a sequence of a nucleic acid molecule used to produce a mature detector fusion molecule is determined. The sequence of the nucleic acid molecule may be a sequence of the gene encoding the protein sub-unit, a randomly determined sequence, or a sequence including, without limitation, a unique site, such as a restriction site on a promotion site, e.g., a T7 promoter sequence. The nucleic acid molecule used in the production of the mature detector fusion molecule may include any type of known nucleic acid molecule including DNA, RNA, a peptide nucleic acid (PNA) molecule, or any combination of nucleic acid molecules thereof. Regardless of the type of nucleic acid molecule selected, as depicted in dialog box 112, a coupling reagent is attached to the nucleic acid molecule, thereby forming a modified nucleic acid molecule. For instance, the detector fusion molecule can identify and quantify disease markers and virulence proteins as target analytes, thereby allowing medical practitioner 10 of FIG. 1 to diagnose disease in patient 12.

As indicated in dialog box 114, the reactive intermediate is placed in contact with the modified nucleic acid, such that a reaction is catalyzed between the reactive moiety of the reactive intermediate and the coupling reagent of the nucleic acid molecule, thereby forming the mature detector fusion molecule. As indicated in dialog box 116, the detector fusion molecule is used to identify and quantify a target analyte. For instance, the detector fusion molecule can identify and quantify disease markers and virulence proteins as target analytes, thereby allowing medical practitioner 10 of FIG. 1 to diagnose disease in patient 12.

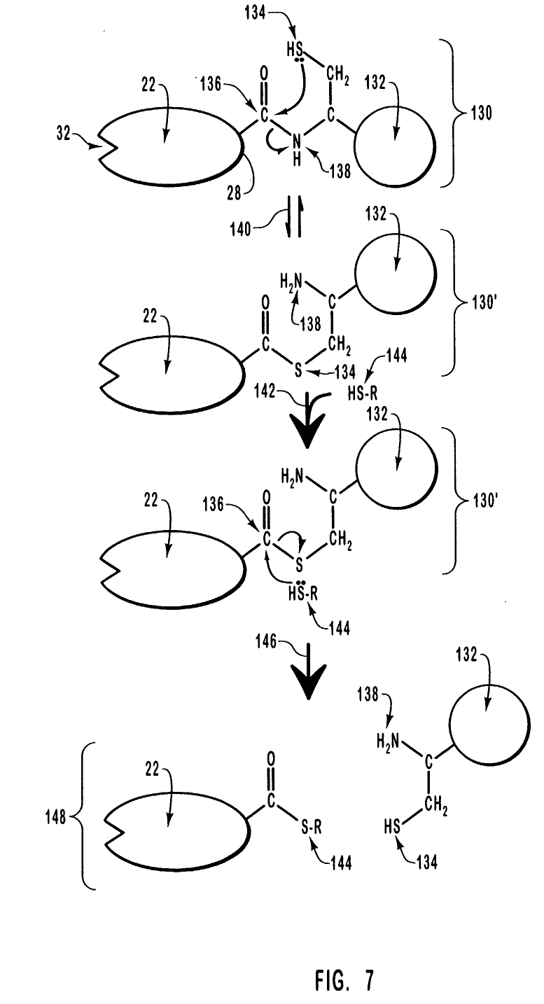

FIG. 7 depicts steps in a method for making a reactive intermediate used to produce a detector fusion molecule. The top of FIG. 7 illustrates a protein sub-unit-intein recombinant protein 130. Protein sub-unit 22 of protein sub-unit-intein recombinant protein 130 of FIG. 7 is a recombinant protein obtained from a recombinant protein sub-unit library as described above with reference to FIG. 6. Protein sub-unit-intein recombinant protein 130 is produced by cloning a gene encoding protein sub-unit 22 into an intein-based cloning vector, such as those available from New England Biolabs of Beverly, Mass., thereby forming a protein sub-unit intein fusion sequence. The intein-based cloning vector including the protein sub-unit intein fusion sequence is over-expressed in a bacterial cell, and protein sub-unit-intein recombinant protein 130 is isolated. Although the illustrated embodiment has been described using an intein-based cloning vector, any other self-splicing cloning vector system may be used in the alternative.

As further illustrated in FIG. 7, an amino sulfur shift occurs when a pair of electrons from a thiol-containing group 134 of protein sub-unit-intein recombinant protein 130 attack a carbonyl carbon 136 of protein sub-unit-intein recombinant protein 130. The electrons from the carbonyl carbon 136 are shifted to an amino moiety 138 of protein sub-unit-intein recombinant protein 130 as illustrated at step 140. The resulting protein sub-unit-intein recombinant protein 130′ is substantially the same as protein sub-unit-intein recombinant protein 130 before the amino sulfur shift, except that amino moiety 138 has shifted places with thiol-containing group 134. The amino sulfur shift is efficient at a pH of approximately 8.0.

At a step 142, an autocatalytic hydrolysis reaction is induced by adding a reactive moiety 144 and tris(2-carboxyethyl)-phosphine (TCEP) to a solution of the protein sub-unit-intein recombinant protein 130′. A pair of electrons from reactive moiety 144 attacks carbonyl carbon 136 of protein sub-unit intein recombinant protein 130′. At a step 146, intein molecule 132, or portion, attached to amino moiety 138 and thiol-containing group 134 are removed from protein sub-unit 22, which remains attached to reactive moiety 144, thereby forming a reactive intermediate 148. Reactive moiety 144 may comprise any thiol-containing group such as thiophenol or mercaptoethanesulfonic acid.

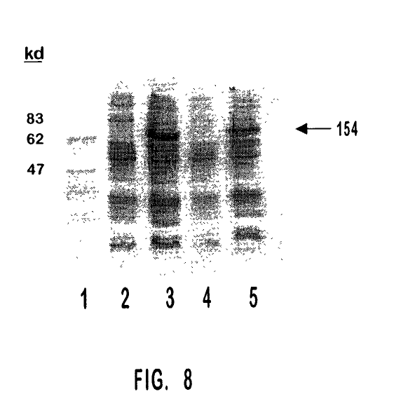

FIG. 8 is a photograph of two protein sub-unit-intein recombinant proteins of the types shown in FIG. 7 on an acrylamide gel and stained with Coomassie Blue. A gene encoding a streptavidin protein molecule is cloned in frame to an amino terminal end of a intein gene, such as a Saccharomyces cerevisiae VMA intein coding sequence, thereby producing a streptavidin-intein fusion sequence. The streptavidin-intein fusion sequence is placed in an expression vector, such as the bacterial expression vector pTYB1, which is under the control of an inducible promoter, and is transformed into a bacteria cell, such as Escherichia coli ER2256. The bacterial cells are grown to an Optical Density (OD) of 0.8 at 600 nm, and expression of the streptavidin-intein fusion sequence is induced using 1 mM isopropyl thiogalactopyranoside (IPTG) for four hours. Bacterial cells are collected by centrifugation, and cell-free lysates of an uninduced streptavidin-intein fusion sequence and an induced streptavidin-intein fusion sequence are fractionated on a 12% acrylamide gel. Acrylamide gel is stained with Coomassie blue, thereby revealing multiple bands of protein as illustrated in FIG. 8.

Lane 1 illustrates a protein size standard. Lane 4 represents the uninduced streptavidin-intein fusion sequence, and Lane 5 represents the induced streptavidin-intein fusion sequence. In Lane 5 a streptavidin-intein protein fusion is present at arrow 154, thus indicating that a streptavidin-intein recombinant protein of the expected site is produced.

Referring to Lanes 2 and 3, a gene coding for a peptide aptamer that binds human cyclin-dependent kinase 2 (hCDK2) is cloned and expressed as described herein with reference to the streptavidin-intein fusion sequence, thereby producing a peptide aptamer-intein fusion sequence. The peptide aptamer-intein fusion sequence is transformed and expressed in a bacterial cell. Lane 3 of FIG. 8 depicts an induced peptide aptamer-intein fusion sequence and Lane 2 depicts an uninduced peptide aptamer-intein fusion sequence. As shown in Lane 3, the induced peptide aptamer-intein fusion sequence produced peptide aptamer-intein fusion protein at the expected size as indicated at arrow 154.

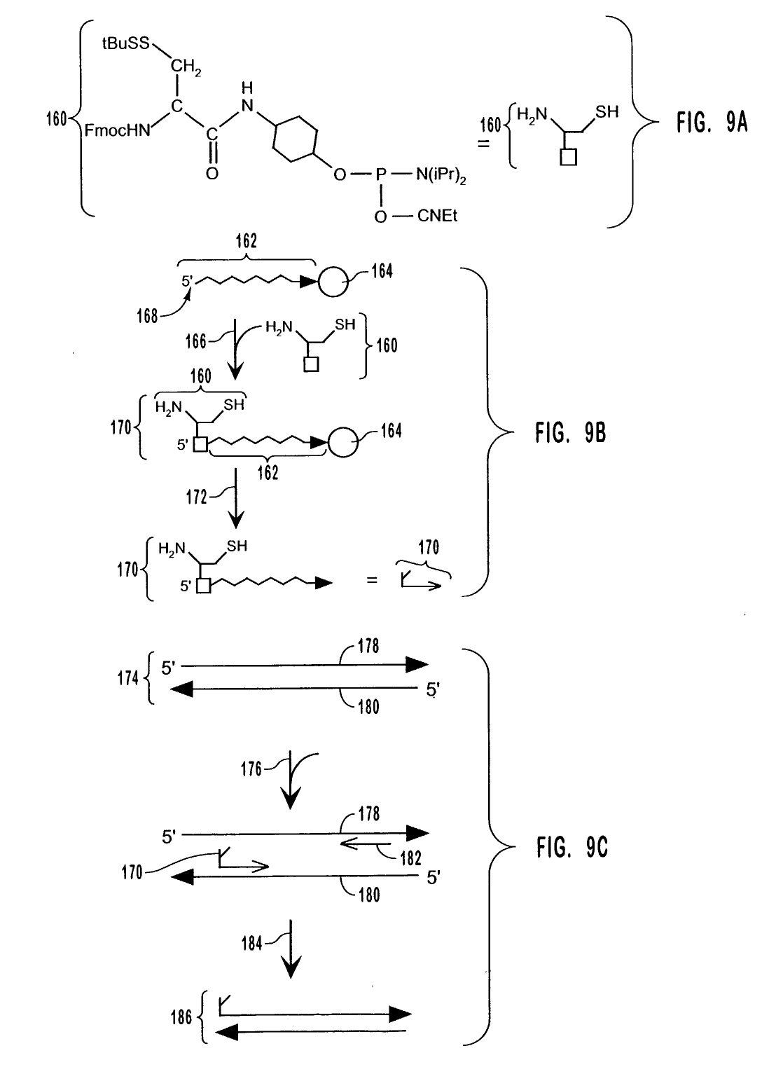

FIG. 9A is a diagram of the molecular structure of a coupling reagent 160, such as a phosphoramidite-containing molecule. Coupling reagent 160 is illustrated in a condensed form on the right side of the equal sign for ease of illustration subsequently in FIGS. 9B and 9C.

FIG. 9B depicts an oligonucleotide 162 attached to a solid phase 164. Oligonucleotide 162 is incorporated into detector fusion molecule 20 of FIG. 2 and comprises nucleic acid molecule 26. Oligonuceotide 162 is manufactured using a conventional solid-phase synthesis process, such as an oligonucleotide synthesizer. At a step 166, coupling reagent 160 is attached to the 5′ end 168 of oligonucleotide 162 by adding coupling reagent 160 to the oligonucleotide synthesizer as oligonucleotide 162 is synthesized, thereby forming an altered oligonucleotide 170 that remains attached to solid phase 164. Since altered oligonucleotide 170 includes a phosphoramidite-containing molecule as coupling reagent 160, altered oligonucleotide 170 is a cysteine-modified oligonucleotide. At a step 172, altered oligonucleotide 170 is removed from solid phase 164. As further depicted in FIG. 9B, altered oligonucleotide 170 is illustrated in a condensed form on the right side of the equal sign for ease of illustration in subsequent diagrams.

FIG. 9C illustrates steps used to attach altered oligonucleotide 170 of FIG. 9B to a double-stranded DNA molecule 174. A PCR reaction is used to incorporate altered oligonucleotide 170 into double-stranded DNA molecule 174. At a step 176, an upper strand 178 and a lower strand 180 of double-stranded DNA molecule 174 are separated, and a primer 182, altered oligonucleotide 170, dNTPs, and DNA polymerase are added to separated strands 178 and 180. Altered oligonucleotide 170 hybridizes to lower strand 180, and altered oligonucleotide 170 is extended by the DNA polymerase, thereby forming a modified nucleic acid molecule 186, which has coupling reagent 160 of FIG. 9A incorporated therein. Modified nucleic acid molecule 186 of FIG. 9C may be used as nucleic acid molecule 26 of detector fusion molecule 20 as shown in FIG. 2.

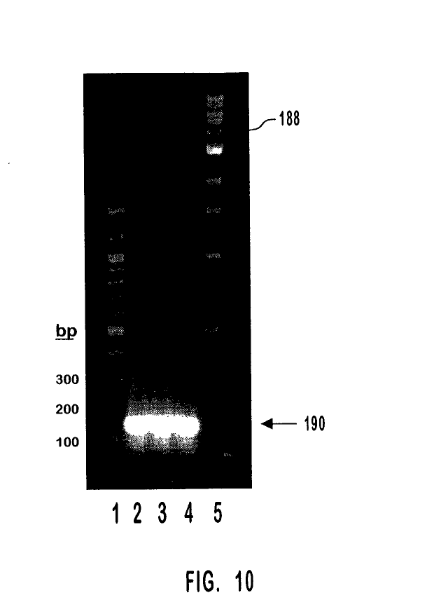

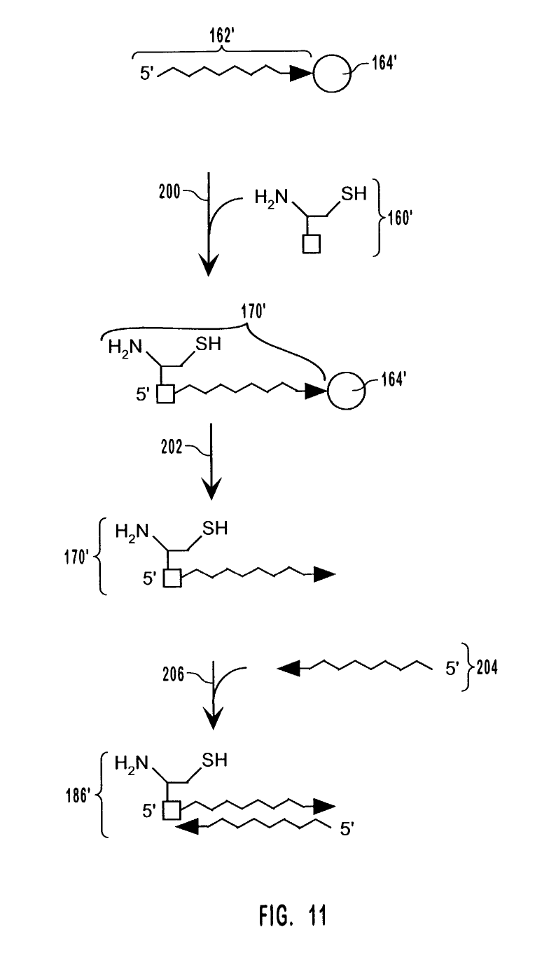

FIG. 10 is a photograph of a first sample of a modified nucleic acid molecule produced using conventional phosphoramidite-based oligonucleotide chemistries, such as the synthesis steps illustrated in FIGS. 9A, 9B, and 9C. In FIG. 10 the modified nucleic acid molecule is resolved on an agarose gel 188 and stained with ethidium bromide. The modified nucleic acid molecule illustrated in FIG. 10 is produced using an oligonucleotide 162, comprising DNA, attached to coupling reagent 160, such as a phospharamidite-containing molecule, thereby forming altered oligonucleotide 170, as illustrated in FIG. 9B. Altered oligonucleotide 170 comprising DNA is used as a primer in a PCR reaction, and added to a mixture comprising double-stranded DNA 174, dNTPs, and DNA polymerase, thereby producing modified nucleic acid molecule 186. Modified nucleic acid molecule 186 comprising double-stranded DNA molecule 174 and coupling reagent 160 is resolved on a 1% agarose gel and stained with ethidium bromide. As illustrated in FIG. 10, modified nucleic acid molecule 186 is indicated on agarose gel 188 at arrow 190.

FIG. 11 is a schematic representation of a second embodiment of a method used to produce a modified nucleic acid molecule 186′. As illustrated at the top of FIG. 11, an oligonucleotide 162′ is attached to a solid phase 164′. At a step 200, a coupling reagent 160′, such as a phosphoramadite-containing molecule, is attached to oligonucleotide 162′, thereby producing altered oligonucleotide 170′ that remains attached to solid phase 164′. At a step 202, altered oligonucleotide 170′ is removed from solid phase 164′. A complementary oligonucleotide 204 is hybridized to altered oligonucleotide 170′, thereby producing modified nucleic acid molecule 186′. Modified nucleic acid molecule 186′ of FIG. 11 may be used as nucleic acid molecule 26 of detector fusion molecule 20 as depicted in FIG. 2.

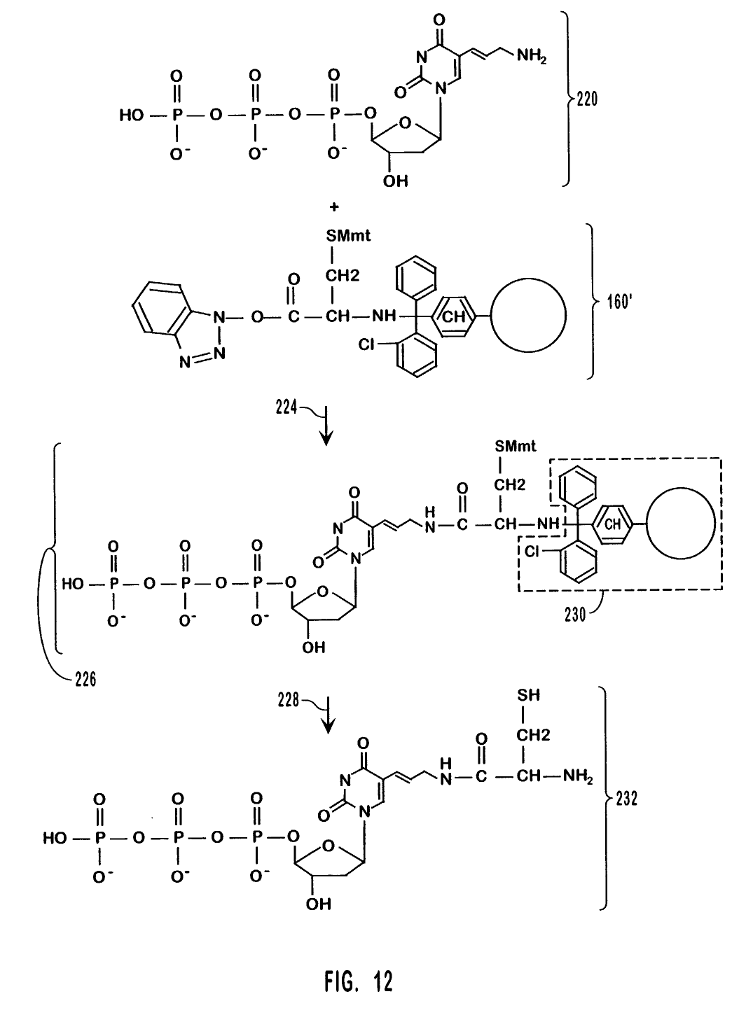

FIG. 12 is a schematic representation of steps in a first embodiment of a method for producing a modified nucleotide. A nucleotide 220 is shown at the top of FIG. 12. In the illustrated embodiment, nucleotide 220 is aminoallyl dUTP, but other nucleotides may be used in the alternative. A coupling reagent 160′ is depicted below nucleotide 220. Coupling reagent 160′ is a N-(2-chlorotrityl polystyrene)-S-p-methoxytrityl-L-cysteine benzotriazoyl ester. At a step 224, nucleotide 220 is attached to coupling reagent 160′ by adding dimethylacetamide, thus producing a nucleotide-coupling reagent complex 226. A portion 230 is cleaved from nucleotide-coupling reagent complex 226 at a step 228 by adding trifluoroacetic acid and dichloromethane, thereby producing a modified nucleotide 232. Although modified nucleotide 232 depicted in FIG. 12 is a cysteine-modified dUTP, other modified nucleotides may be produced from other nucleotides.

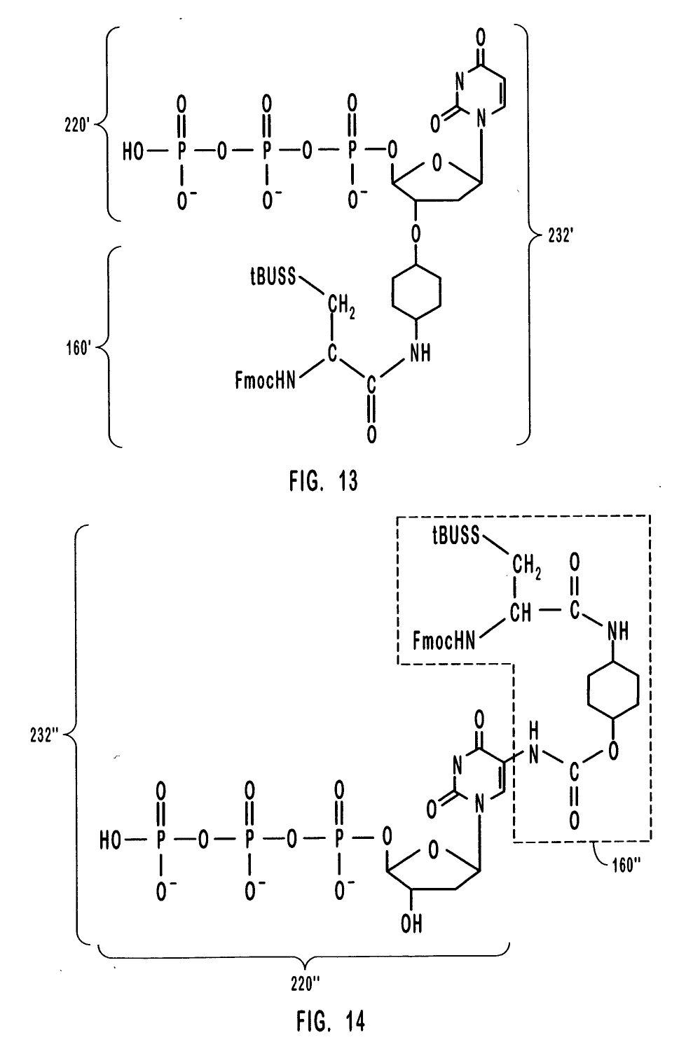

FIG. 13 and FIG. 14 illustrate molecular structures of alternative embodiments of modified nucleotides incorporating teachings of the present invention. FIG. 13 depicts a second modified nucleotide 232′ including a nucleotide 220′ attached to coupling reagent 160′. FIG. 14 illustrates a third modified nucleotide 232″ comprising a nucleotide 220″ attached to a coupling reagent 160″. Modified nucleotides 232, 232′, and 232″ shown in FIGS. 12, 13, and 14, respectively, have coupling reagents 160, 160′, and 160″ attached at different locations on the nucleotide portion of the modified nucleotide.

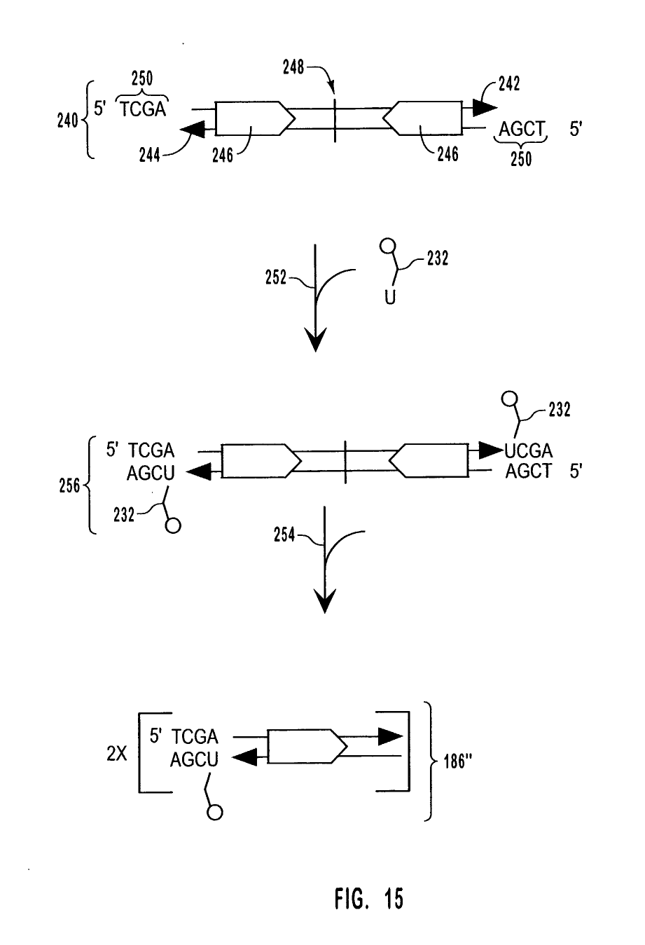

FIG. 15 is a schematic representation of steps in a first embodiment of a method incorporating teachings of the present invention used to incorporate a modified nucleotide 232, such as the modified nucleotide 232 of FIG. 12, into a nucleic acid molecule, thereby to form a third embodiment of a modified nucleic acid molecule. At the top of FIG. 15 is depicted a symmetrical nucleic acid molecule 240. Symmetrical nucleic acid molecule 240 includes a top strand 242 and a bottom strand 244. Symmetrical nucleic acid molecule 240 is DNA, but may be RNA, or a combination of RNA and DNA in alternative embodiments. Symmetrical nucleic acid molecule 240 includes a restriction enzyme site 248, a pair of T7 promoter sequences 246, a pair of nucleotide overhangs 250. Top strand 242 and bottom strand 244 include nucleotide overhangs 250 that are produced by cutting symmetrical nucleic acid molecule 240 with a restriction enzyme, such as XhoI.

At a step 252, DNA polymerase, nucleotides, and a modified nucleotide 232 are added to symmetrical nucleic acid molecule 240 in such a manner that added nucleotides fill in nucleotide overhangs 250. In the illustrated embodiment, modified nucleotide 232 is cysteine-modified dUTP, and added nucleotides include dATP, dCTP, and dGTP. Since nucleotide overhangs 250 comprise the sequence TCGA, modified nucleotide 232 will be incorporated into symmetrical nucleic acid molecule 240 at each end complementary to the adenine A in each nucleotide overhang 250. The dATP, dCTP, and dGTP will be incorporated into symmetrical nucleic acid molecule 240, thereby forming a modified symmetrical nucleic acid molecule 256. At a step 254, a restriction enzyme is used to sever modified symmetrical nucleic acid molecule 256 at restriction enzyme site 248 into two modified nucleic acid molecules 186″ or fragments. Since restriction enzyme site 248 is located substantially equidistant from the pair of nucleotide overhangs 250, the resulting pair of modified nucleic acid molecules 186″ are substantially identical. Restriction enzyme site 248 may be an EcoRI site, wherein the restricting enzyme EcoRI is used for severing modified symmetrical nucleic acid molecule 256.

In an alternative embodiment, a modified nucleotide may be attached to a double-stranded nucleic acid molecule by nicking one end of the double-stranded nucleic acid molecule with a restriction enzyme, thereby producing a nicked double-stranded nucleic acid molecule with a terminal nucleotide removed from one end of the nicked double-stranded nucleic acid molecule. The modified nucleotide may be attached to the nicked double-stranded nucleic acid using a fill-in reaction, thereby creating a modified nucleic acid molecule.

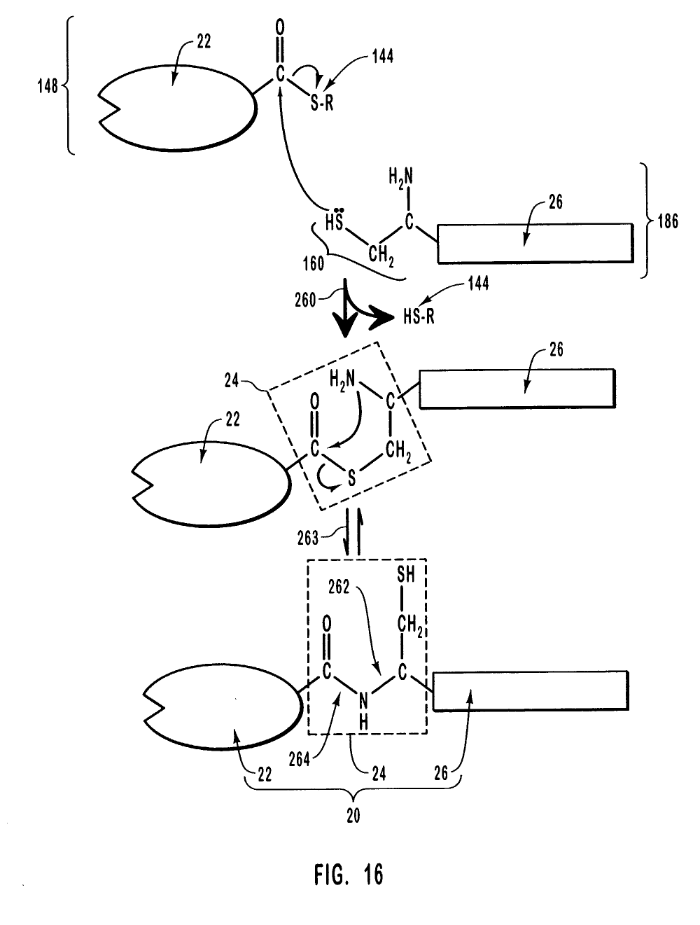

FIG. 16 is a schematic representation of steps in a method used to attach reactive intermediate 148 of FIG. 7 to a modified nucleic acid molecule, such as modified nucleic acid molecules 186, 186′, and 186″ depicted in FIGS. 9, 11, and 15, respectively. As previously described herein, reactive intermediate 148 includes protein sub-unit 22 attached to reactive moiety 144, and modified nucleic acid molecule 186 comprises nucleic acid molecule 26 attached to coupling reagent 160. As illustrated, coupling reagent 160 comprises a cysteine-like moiety, because coupling reagent 160 resembles a side chain of the amino acid, cysteine.

At a step 260, reactive moiety 144 is displaced from reactive intermediate 148 by coupling reagent 160 of modified nucleic acid molecule 186, thereby forming linker 24 between protein sub-unit 22 and nucleic acid molecule 26. The displacement of reactive moiety 144 from protein sub-unit 22 results in the formation of a covalent bond between protein sub-unit 22 and coupling reagent 160 of modified nucleic acid molecule 186. An N-S acyl shift takes place at step 263, thereby producing a peptide bond 262 and resulting in mature detector fusion molecule 20. An amide bond 264 is also produced in linker 24 as a result of the N-S acyl shift, thereby resulting in linker 24 including an amide linkage.

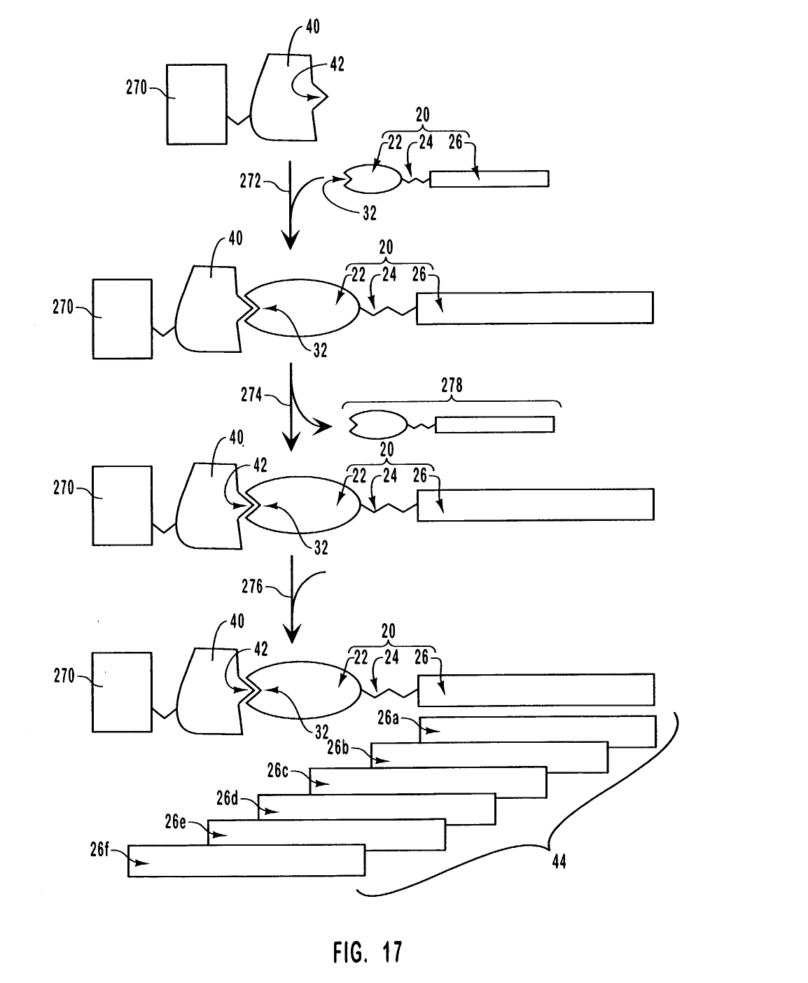

FIG. 17 is a schematic representation of a second embodiment of a method for binding a target analyte 40 of a sample, and amplifying detector fusion molecule 20 bound to target analyte 40. Target analyte 40 of the sample is immobilized on a solid substrate 270, such as the side of a microfuge tube or a microtiter plate. At a step 272, target analyte 40 is contacted with detector fusion molecule 20. Although FIG. 17 depicts one detector fusion molecule 20 for ease of illustration, it will be apparent that a quantity of detector fusion molecules 20 may be placed in contact with a plurality of various target analytes 40 within the sample.

Binding site 32 of protein sub-unit 22 of detector fusion molecule 20 specifically binds to epitope 42 of target analyte 40. At a step 274, any unbound detector fusion molecules 20 are washed away from the sample, thereby leaving only detector fusion molecules 20 that are specifically bound to target analytes 40. Nucleic acid molecule 26 of the detector fusion molecule 20 is amplified at step 276, thereby producing amplification product 44.

The type of nucleic acid molecule 26 used to produce detector fusion molecule 20 dictates, at least in part, a type of method used to produce the amplification product 44. For instance, if nucleic acid molecule 26 includes a T7 promoter sequence, then T7 polymerase and rNTPs could be used to amplify nucleic acid molecule 26. Alternatively, if nucleic acid molecule 26 comprises DNA, then PCR may be used to amplify nucleic acid molecule 26, wherein a primer complementary to a portion of the sequence of nucleic acid molecule 26 of detector fusion molecule 20 bound to target analyte 40 is hybridized to nucleic acid molecule 26. For PCR amplification, DNA-thermostable polymerase and dNTPs are added to produce amplification product 44. In the alternative, if nucleic acid molecule 26 of bound detector fusion molecule 20 is a hybrid RNA-DNA molecule, then RNAse may be added, thereby releasing the single-stranded DNA molecule, which may be directly amplified using PCR as previously described herein. Alternatively, the single-stranded DNA molecule may be converted to double-stranded DNA using Klenow polymerase, or subsequently amplified using T7 RNA polymerase and rNTPs.

Amplification product 44 is identified by resolving amplification product 44 on a gel, such as agarose or polyacrylamide, and staining amplification product 44 with SYBR green or ethidium bromide. In yet another alternative embodiment, amplification product 44 may be identified by sequencing a nucleotide sequence of amplified nucleic acid molecules 26a through 26f using known processes.

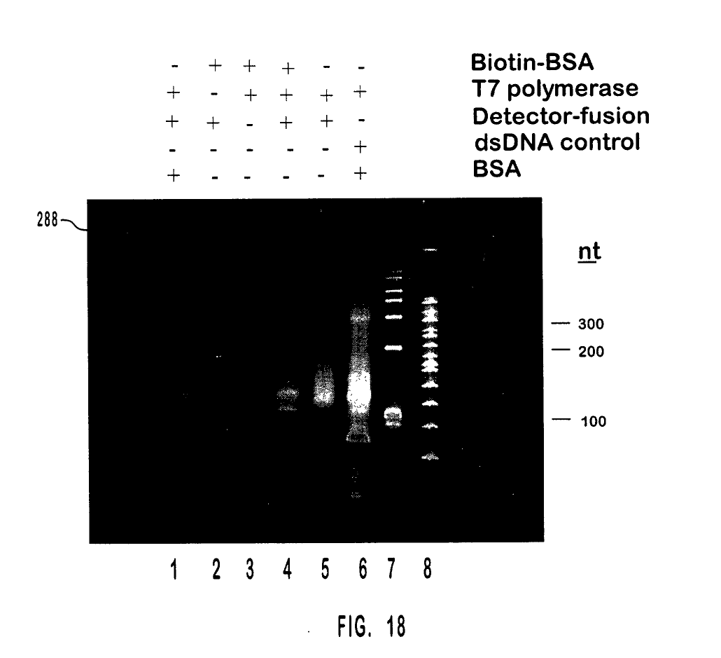

FIG. 18 is a photograph illustrating streptavidin detector fusion molecules identifying biotin. Streptavidin detector fusion molecule is produced using the steps described herein with reference to FIG. 16. Streptavidin-intein protein fusion molecule of FIG. 18 is autocatalytically spliced to remove intein molecule, thereby producing reactive intermediate that comprises a streptavidin protein sub-unit and reactive moiety. A modified nucleic acid molecule comprising a T7 promoter sequence is designed specifically for streptavidin protein sub-unit. Modified nucleic acid molecule is attached to reactive intermediate as described herein with reference to FIG. 16, thereby producing streptavidin detector fusion molecule.

To detect the ability of streptavidin detector fusion molecule to bind biotin, biotinylated-BSA or BSA alone are used as target analytes. Since streptavidin is known to bind specifically to biotin, streptavidin detector fusion molecule works as an example to illustrate a detector fusion molecule produced using the methods of the present invention binding a target analyte. Biotinylated-BSA or BSA alone is non-specifically adsorbed to wells of a polystyrene ELISA plate, and surfaces of ELISA plate not adsorbed with biotinlyated-BSA or BSA alone are blocked with 0.2% casein. Streptavidin detector fusion molecule is added to loaded wells, thus allowing streptavidin detector fusion molecules to bind biotin adsorbed to wells of the ELISA plate. Unbound streptavidin detector fusions are removed from wells by extensive washing.

The presence of bound streptavidin detector fusion molecules is detected by adding T7 polymerase, rNTPs, and buffer to the wells to amplify nucleic acid molecules of bound streptavidin detector fusion molecules. RNA synthesis is conducted for four hours. Amplification products of the RNA synthesis are fractionated on a 10% acrylamide, 50% urea denaturing gel 288, and stained with SYBR green II, an agent used to stain single-stranded RNA, thereby illuminating amplification products. Amplification products are illustrated in FIG. 18.

Negative controls are present in each of Lane 1 that lacks biotin-BSA, in Lane 2 that lacks T7 polymerase, and in Lane 3 that lacks the streptavidin detector fusion. As seen in Lanes 1, 2, and 3, no significant amplification product was generated. Lane 5 includes T7 polymerase and streptavidin detector fusions, and Lane 6 is a positive control including T7 polymerase, a double-stranded DNA sequence including a T7 promoter sequence, and BSA. As shown in Lanes 5 and 6, an RNA amplification product is generated. Lane 4 includes biotinylated-BSA, T7 polymerase, and streptavidin detector fusion molecules. After amplification, Lane 4 is depicted as producing an amplification product, thus demonstrating that streptavidin detector fusion molecules bind specifically to biotin, and that nucleic acid molecules of streptavidin detector fusion molecules are amplifiable.

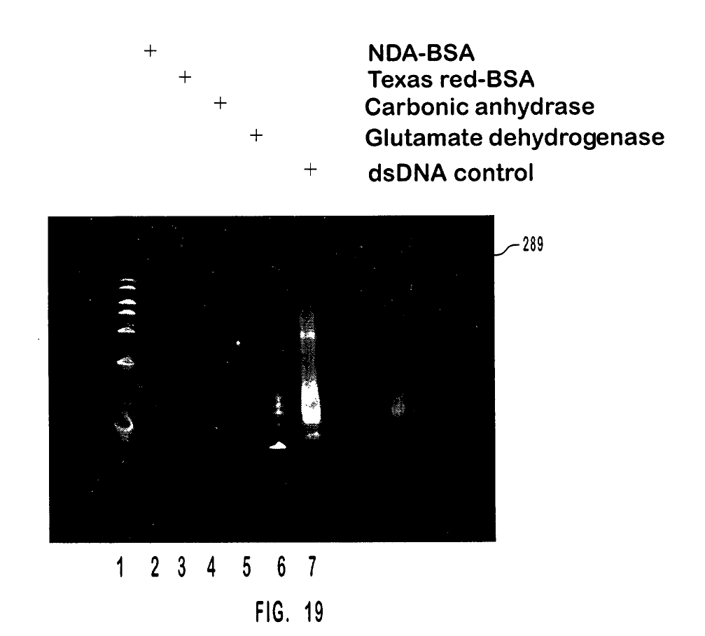

FIG. 19 is another photograph illustrating the use of streptavidin detector fusion molecules in the same manner as described herein with reference to FIG. 18. The experiment of FIG. 19 is conducted in concert with the experiment of FIG. 18. In FIG. 19, instead of using biotinylated-BSA as target analyte, target analytes of the experiment of FIG. 18 included NDA-BSA, Texas red-BSA, carbonic anhydrase, and glutamate dehydrogenase. The results of interrogation and subsequent amplification of target analytes with streptavidin detector fusion molecules are shown on gel 289 in Lanes 2, 3, 4, and 5. No significant amplification products are produced, thus demonstrating that the binding and subsequent amplification of the streptavidin detector fusion molecules are specific for biotin.

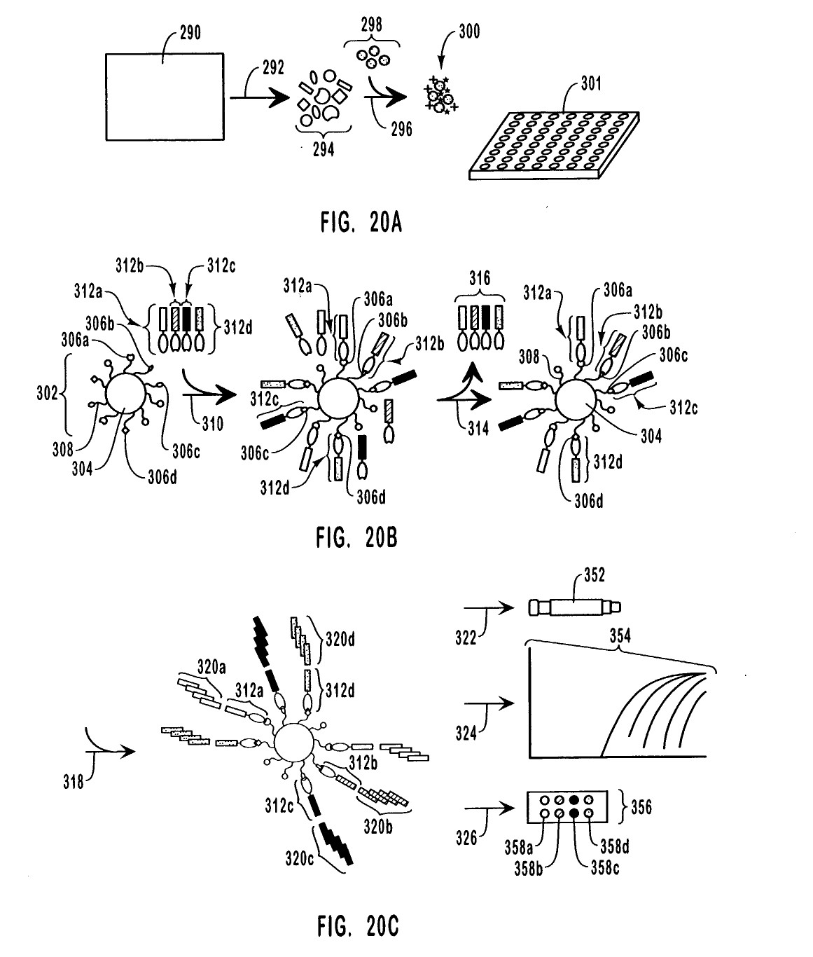

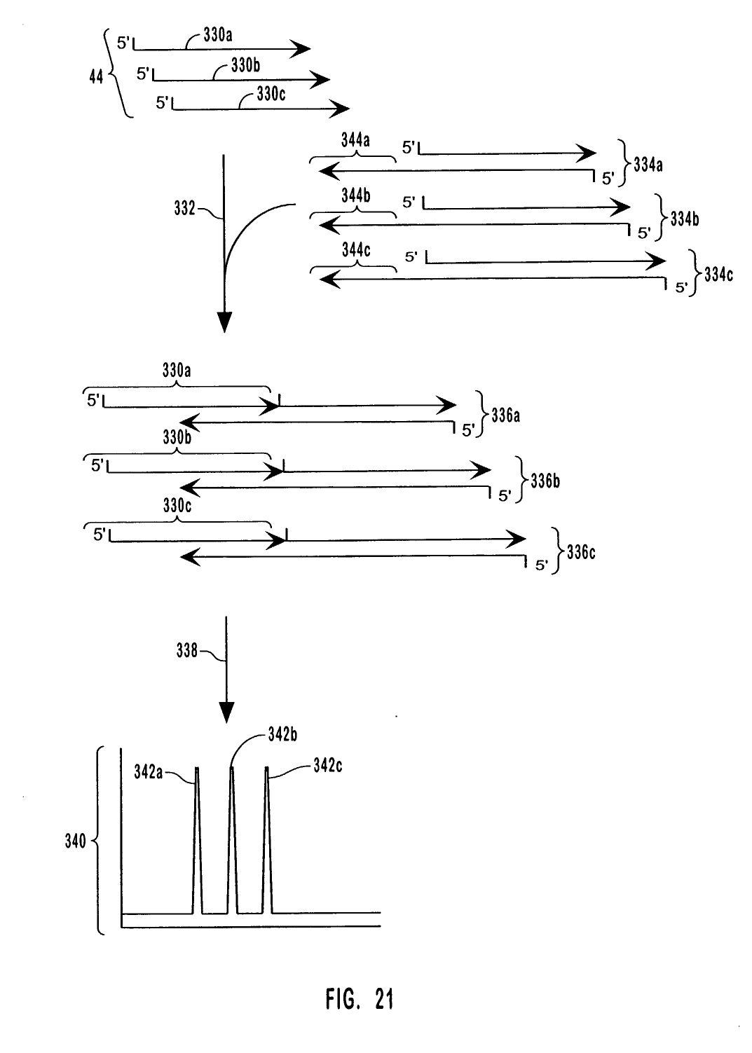

FIGS. 20A, 20B, and 20C taken together illustrate a method of detecting and identifying target analytes in sample 290. At a step 292 constituents of sample 290 are disrupted into a variety of sample molecules 294. At a step 296, sample molecules 294 are contacted with a substrate, such as magnetic micro-particles 298, thereby covalently coupling and immobilizing sample molecules 294 to magnetic micro-particles 298. Since magnetic micro-particles 298 have attractive properties attributed to a surface thereof as illustrated with stars and plus symbols 300, sample molecules 294 are bound to magnetic micro-particles 298 using known processes. In addition to using magnetic micro-particles 298 as a substrate to bind sample molecules 294, an ELISA plate 301 may be used as the substrate to bind sample molecules 294.

An immobilized sample 302 including an individual magnetic micro-particle 304 and a number of individual sample molecules, or target analytes 306a through 306d, are illustrated in FIG. 20B. A covalent bond 308 is shown binding individual magnetic micro-particle 304 to various target analytes 306a through 306d. At step 310, different detector fusion molecules 312a, 312b, 312c, and 312d are placed in contact with individual magnetic micro-particle 304 with various target analytes 306a through 306d bound thereto. Detector fusion molecules 312a through 312d specifically bind to target analyte 306a through 306d for which detector fusion molecule 312 has been designed to specifically bind. Any unbound detector fusion molecules 316 are washed away from bound detector fusion molecules 312a through 312d at a step 314.

At a step 318, nucleotides and a polymerase, such as a DNA or an RNA-polymerase, are added to bound detector fusion molecules 312a through 312d, such that polymerase will amplify nucleic acid molecule 26 of bound detector fusion molecules 312a through 312d, thereby producing amplification products 320a through 320d for bound detector fusion molecules 312a through 312d. Amplification products 320a through 320d are identified or quantified using various known methods. Identification of the amplification products 320a through 320d allows medical practitioner 10 of FIG. 1 to identify the illness of patient 12.

In a first method, amplification products 320a though 320d are passed through a HPLC column 352, such that amplification products 320a through 320d may be separated on a basis of chemical composition.

In a second method indicated at a step 324, amplification products 320a through 320d are processed with quantitative PCR 354 using a plurality of detection techniques, such that after a specified number of rounds of PCR, PCR products and quantitated by measuring an amount of radioactivity or fluorescence emitted by detectable markers. Other detectable markers that may be used include intercalating fluorescent dyes, such as Hoescht 33342, that are detectable with fluorescence microscopes.

In a third method displayed at a step 326, amplification products 320a through 320d are placed on a hybridization microarray 356 or chip. As depicted in FIG. 20C, hybridization chip includes four spots 358a through 358d. Each spot 358a through 358d has a number of probes, such as single-stranded nucleic acid molecules attached thereto comprising a sequence complementary to a sequence of amplification products 320a through 320d, such that single-stranded amplification products 320a through 320d will hybridize to attached single-stranded nucleic acid molecules of spots 358a through 358d. Once amplification products 320a through 320d hybridize to probes of spots 358a through 358d, an amount of amplification products 320a through 320d hybridized to probes is measured. Based on the amount of hybridized amplification products 320a through 320d, an amount of target analytes 306a through 306d of sample 290 is determined.

An example of a method used to detect an amplification product produced by amplifying a nucleic acid molecule of a detector fusion molecule is illustrated in FIG. 21. In this example, amplification product 44 includes three single-stranded nucleic acid molecules 330a through 330c. At a step 332, three different detector nucleic acid molecules 334a through 334c are added to single-stranded nucleic acid molecules 330a through 330c. As illustrated, three different detector nucleic acid molecules 334a through 334c are of different lengths, wherein detector nucleic molecule 334a is the shortest and detector nucleic molecule 334c is the longest. Single-stranded nucleic acid molecules 330a through 330c of amplification product 44 hybridize to detector nucleic acid molecules 334a through 334c, thereby forming hybridized duplexes 336a through 336c. Hybridized duplexes 336a through 336c are subjected to capillary electrophoresis at step 338, where hybridized duplexes 336a through 336c are resolved on a basis of size.

A capillary electrophoresis chromatogram 340 is generated, and peaks 342a through 342c of capillary electrophoresis chromatogram 340 indicate the presence of hybridized duplexes 336a through 336c. In this embodiment, detector nucleic acid molecules 334a through 334c maybe used to distinguish single-stranded nucleic acid molecules 330a through 330d on a basis of sequence because the single-stranded overhangs 344a through 344c of hybridized duplexes 336a through 336c each have a different nucleotide sequence. The different nucleotide sequences of single-stranded overhangs 344a through 344c are designed to specifically hybridize to a sequence of each of the single-stranded nucleic acid molecules 330a through 330c. In this manner, since hybridized duplexes 336a through 336c are of different lengths, peaks 342a through 342c of capillary electrophoresis chromatogram 340 are used to distinguish each of single-stranded nucleic acid molecules 330a through 330c on a basis of size.

In another embodiment, amplification products 320a through 320d produced with a primer labeled with a detectable marker are resolved on a gel, thereby forming bands of amplification products 320a through 320d separated on a basis of size. An intensity of bands of amplification products 320a through 320d is measured using known processes, such as using a phosphoimager to measure radioactivity emitted by detectable markers of primers.

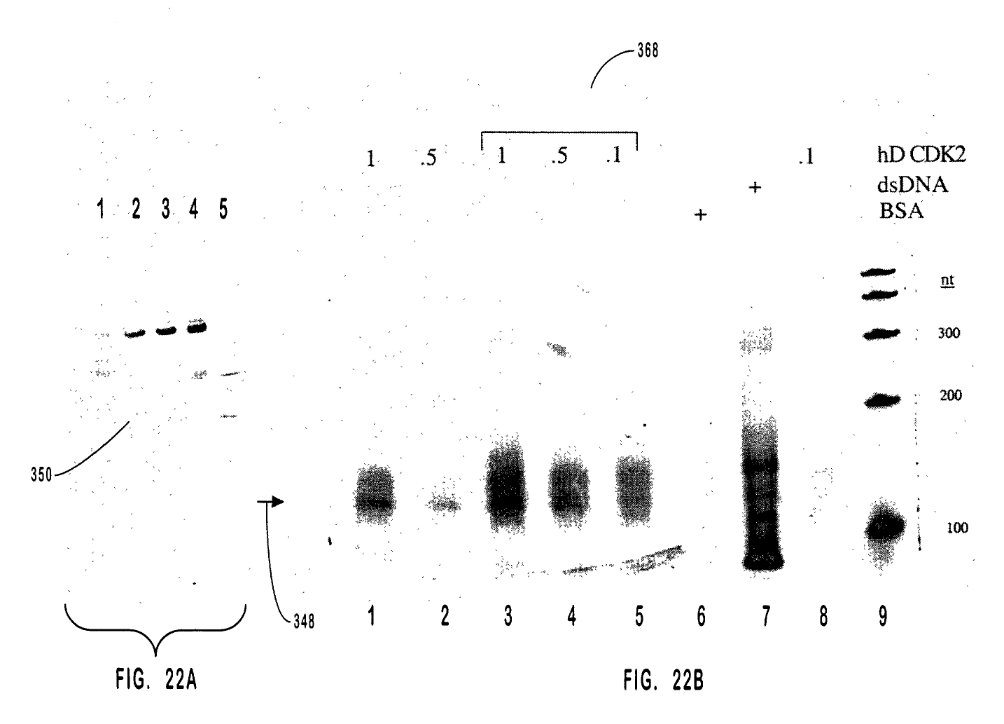

As an example of how detector fusion molecules of the present invention are used to quantify an amount of a target analyte, reference is made to FIG. 22A. FIG. 22A is a photograph 350 illustrating samples of recombinant human cyclin-dependent kinase 2 (hCDK2) produced and purified in vitro. The samples are resolved on a denaturing 12% PAGE gel and stained with Coomassie blue. Lane 1 is a protein size standard. Lanes 2 and 3 are ion-exchange purified samples of rhCDK2 and Lane 4 is a crude preparation of rhCDK2. The samples illustrated in FIG. 22A are used as target analytes in the following example.

FIG. 22B is a photograph of amplification products generated using T7 polymerase and detector fusion molecules designed to bind hCDK2 target analyte of FIG. 22A. The example described herein with reference to FIG. 22B was performed in a manner substantially the same as the example described herein with reference to FIGS. 18 and 19. Detector fusion molecules of FIG. 22B comprise a protein sub-unit selected to specifically bind hCDK2 and a nucleic acid molecule including a T7 promoter. The hCDK2 target analyte of FIG. 22A was diluted and non-specifically absorbed to the wells of an ELISA plate. Detector fusion molecules specific for the hCDK2 target analyte were allowed to bind to the hCDK2 target analytes absorbed in the wells, and unbound detector fusion molecules were washed away.

T7 RNA polymerase and rNTPs are added to ELISA plate wells, thereby amplifying nucleic acid molecules of detector fusion molecules bound to the adsorbed hCDK2 target analytes, thereby producing an amplification product. Amplification product is resolved on a denaturing PAGE gel of 10% acrylamide and 50% urea and stained with SYBR green II. A photograph 368 of the resolved amplification product is depicted in FIG. 22B. Bands of stained nucleic acid molecules are observed at arrow 348, which correspond to a length of nucleic acid molecule of detector fusion molecule designed to bind the hCDK2 target analytes. Lane 6 is an amplification product of a negative control with BSA and represents a background of the assay, while Lane 7 is a positive control of an amplification product that includes a double-stranded DNA molecule with a T7 promoter sequence. Lanes 3, 4 and 5 represent amplification products obtained from wells with various concentrations of purified hCDK2 adsorbed therein, wherein an amplification product of Lane 3 has 1 μg of hCDK2 per well, an amplification product of Lane 4 has 0.5 μg of hCDK2 per well, and an amplification product of Lane 5 has 0.1 μg of hCDK2 per well. Lanes 1, 2 and 8 include the same amplification products as Lanes 3, 4 and 5, respectively. Lane 9 is an RNA size standard.

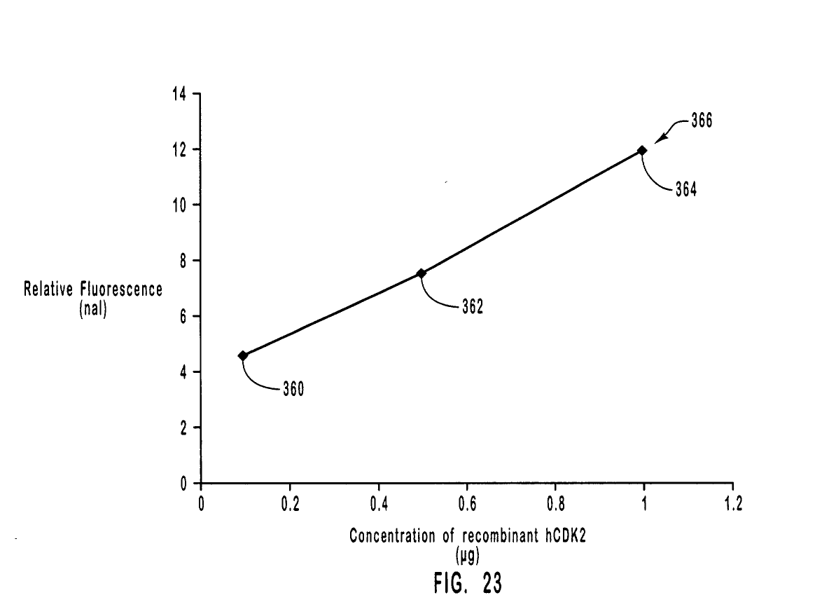

An amount of the amplification product observed on photograph 368 of FIG. 22B is quantified in FIG. 23. FIG. 23 is a graph plotting a relative fluorescence of amplification products of Lanes 3, 4, and 5 of FIG. 22B on a Y-axis versus a concentration of hCDK2 target analyte on a X-axis in the wells used to obtain the amplification product of Lanes 3, 4, and 5 of FIG. 22B. Relative fluorescence of the graph is obtained by scanning the gel of FIG. 22B with a laser tuned at 450 nm, and a fluorescent emission of amplification product was quantified with a photo-multiplier tube, such as Storm 860. A first point 360 illustrates relative fluorescence of amplification product of Lane 5 produced from 0.1 μg of hCDK2 target analyte, a second point 362 indicates relative fluorescence of amplification product of Lane 4 produced from 0.5 μg of hCDK2 target analyte, and a third point 364 represents relative fluorescence of amplification product of Lane 2 produced from 1.0 μg of hCDK2 target analyte. A line 366 connecting points 360, 362, and 364 illustrates that the fluorescence of amplification products increases in a substantially linear fashion indicating that the amount of relative fluorescence correlates with the amount of target analyte sampled.

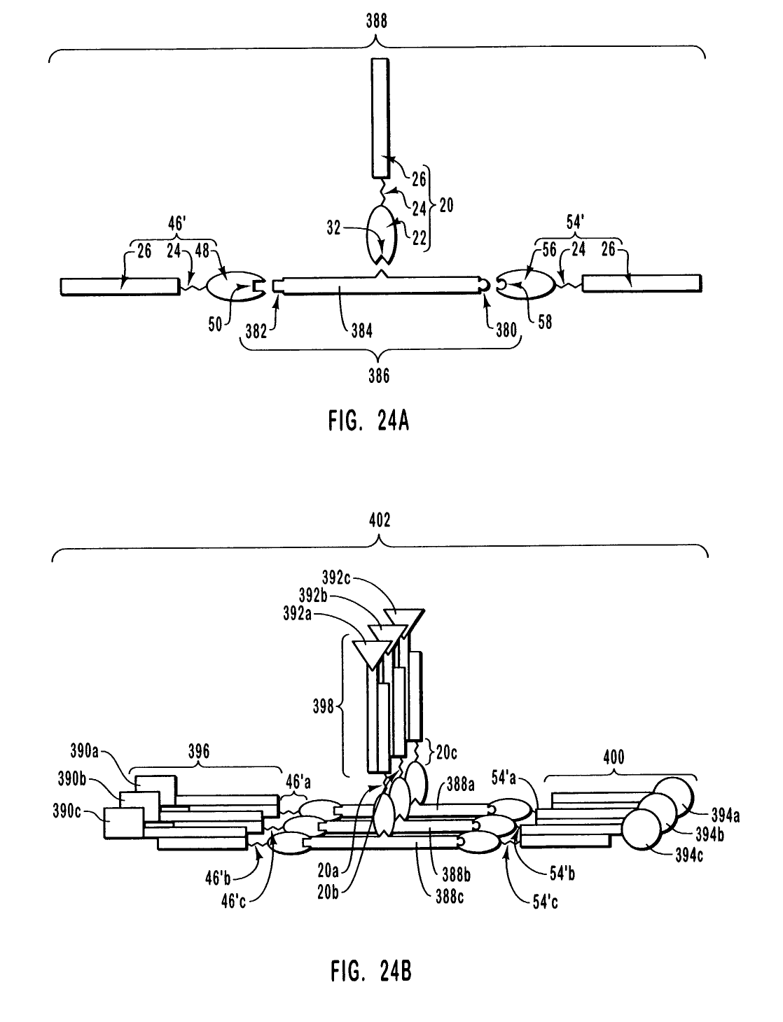

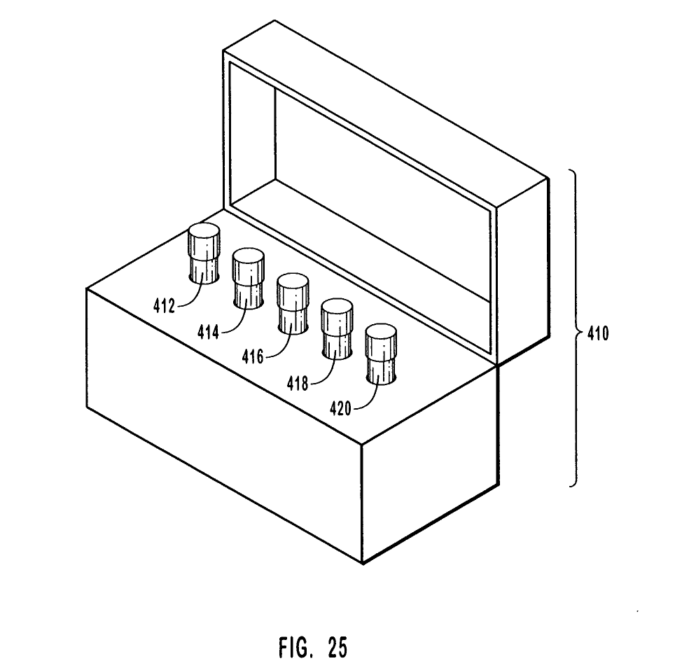

Referring in conjunction to FIGS. 24A and 24B, there is shown a schematic representation of detector fusion molecules used to build a nanostructure. Three detector fusion molecules 20,46′, and 54′ are illustrated. Detector fusion molecules 20,46′, and 54′ are substantially the same as the three detector fusion molecules of FIG. 4, except detector fusion molecules 20,46′, and 54′ of FIG. 24A each include the same nucleic acid molecule 26. A substrate 384 is depicted with epitopes 42, 380, and 382 attached thereto, thereby forming a multimeric complex 386. Epitopes 42, 380, and 382, or target analytes, are attached to substrate 384 for the purpose of constructing a fusion molecule-analyte complex 388. The fusion molecule-analyte complex 388 has utility in the construction of a nanostructure.

Fusion molecule-analyte complexes 388a through 388c are depicted in FIG. 24B. Although three fusion molecule-analyte complexes 388a through 388c are displayed, a plurality of any number of fusion molecule-analyte complexes 388 may be organized in a nanostructure 402. First higher order structures 390a, 390b, and 390c are shown linked to detector fusion molecules 46′a, 46′b, and 46′c. A length of first higher order structure 390a is illustrated with bracket 396. Second higher order structures 392a, 392b, and 392c are depicted linked to detector fusion molecules 20a, 20b, and 20c. A length of second higher order structure 392a is shown with bracket 398. Third higher order structures 394a, 394b, and 394c are depicted bound to detector fusion molecules 54′a, 54′b, and 54′c. A length of third higher structure 394a is displayed with bracket 400.

Detector fusion molecules of the present invention are illustrated in FIG. 25 included in a kit 410. Kit 410 includes a first tube 412 with a detector fusion molecule, a second tube 414 containing a first means for amplifying detector fusion molecule, and a third tube 416 including a second means for visualizing an amplification product generated by first means. Referring again to FIG. 1, kit 410 has utility in testing the blood sample of patient 12, thereby allowing medical practitioner 10 to diagnose the illness of patient 12.

Detector fusion molecules of first tube 412 are of the type of detector fusion molecules 20 described herein with reference to FIG. 2. The contents of second tube 414 will vary depending on the type of nucleic acid molecule 26 used to construct detector fusion molecule 20 of first tube 412. For instance, if nucleic acid molecule 26 includes a T7 promoter sequence, then second tube 414 includes an RNA polymerase, such as T7 RNA polymerase. In this embodiment, a fourth tube 418, including rNTPs, is also included with kit 410. A ribonucleic acid primer may also be included in first means of second tube 414.

In an alternative embodiment, second tube 414 may comprise a DNA polymerase and a DNA primer. DNA primer may have a detectable marker attached thereto. In this alternative embodiment, fourth tube 418 containing dNPTs will be included in kit 410. DNA polymerases that are used include, without limitation, Klenow fragment, Tax polymerase, Vent polymerase, and Deep Vent polymerase.

In any of kit 410 embodiments, second means of third tube 416 includes a stain for visualizing the amplification product. Stains that may be used include ethidium bromide and SYBR green II. In an alternative embodiment, a fifth tube 420 containing a buffer solution for providing optimal binding conditions of detectable fusion molecule marker to target analytes are included.

The invention maybe embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for producing a fusion molecule capable of use as a detector molecule for binding a predetermined target analyte, said method comprising the steps of:
(a) attaching a reactive moiety to a first end of a protein sub-unit, thereby creating a reactive intermediate with said reactive moiety at a first end thereof;
(b) bonding a coupling reagent to a first end of a nucleic acid molecule, thereby forming a modified nucleic acid molecule, said coupling reagent of said modified nucleic acid molecule being capable of displacing said reactive moiety of said reactive intermediate; and
(c) catalyzing a reaction between said reactive moiety of said reactive intermediate and said coupling reagent of said modified nucleic acid molecule, wherein in said reaction:
(i) said reactive moiety is displaced from said first end of said reactive intermediate; and
(ii) a covalent bond is formed between said first end of said reactive intermediate and said first end of said modified nucleic acid molecule.

2. A method as recited in claim 1, wherein said step of attaching comprises the steps of:
(a) cloning a first nucleic acid sequence encoding said protein sub-unit into a vector having a second nucleic acid sequence encoding for an intein segment, thereby producing a protein sub-unit-intein fusion sequence;
(b) expressing said protein sub-unit-intein fusion sequence, thereby producing a protein sub-unit-intein recombinant protein; and
(c) adding said reactive moiety and tris-(2-carboxyethyl) phosphine to a solution containing said protein sub-unit-intein recombinant protein, thereby inducing a hydrolysis reaction wherein:
(i) the intein portion of said protein sub-unit-intein recombinant protein is removed therefrom; and
(ii) said reactive moiety becomes attached to said first end of said protein sub-unit.

3. A method as recited in claim 1, wherein said first end of said protein sub-unit comprises a carboxyl terminus of said protein sub-unit.

4. A method as recited in claim 1, wherein said reactive moiety comprises a thiol-containing group.

5. A method as recited in claim 1, wherein said protein sub-unit comprises a natural protein.

6. A method as recited in claim 1, wherein said protein sub-unit comprises a recombinant peptide aptamer.

7. A method as recited in claim 1, wherein said protein sub-unit comprises a synthetic peptide.

8. A method as recited in claim 1, wherein said covalent bond comprises an amide bond.

9. A method as recited in claim 1, wherein a sequence of said nucleic acid molecule is a barcode for said protein sub-unit.

10. A method for producing a fusion molecule capable of use as a detector molecule for binding a predetermined target analyte, said method comprising the steps of:
(a) attaching a reactive moiety to a first end of a protein sub-unit, thereby creating a reactive intermediate with said reactive moiety at a first end thereof;
(b) bonding a phosphoramidite-containing molecule to a first end of a nucleic acid molecule, thereby forming a modified nucleic acid molecule, said phosphoramidite-containing molecule of said modified nucleic acid molecule being capable of displacing said reactive moiety of said reactive intermediate; and
(c) catalyzing a reaction between said reactive moiety of said reactive intermediate and said phosphoramidite-containing molecule of said modified nucleic acid molecule, wherein in said reaction:
(i) said reactive moiety is displaced from said first end of said reactive intermediate; and
(ii) a covalent bond is formed between said first end of said reactive intermediate and said first end of said modified nucleic acid molecule.

11. A method as recited in claim 10, wherein said step of bonding comprises the steps of:
(a) synthesizing said nucleic acid molecule using an oligonucleotide synthesizer;
(b) adding said phosphoramidite-containing molecule to said oligonucleotide synthesizer; and
(c) incorporating said phosphoramidite-containing molecule on said first end of said nucleic acid molecule.

12. A method as recited in claim 10, wherein said first end of said nucleic acid molecule comprises the 5′ terminus of said nucleic acid molecule.

13. A method as recited in claim 10, wherein said reactive moiety comprises a thiol-containing group.

14. A method as recited in claim 10, wherein said nucleic acid molecule comprises a deoxyribonucleic acid molecule.

15. A method as recited in claim 14, wherein said deoxyribonucleic acid molecule comprises a double-stranded deoxyribonucleic acid molecule.

16. A method as recited in claim 10, wherein said nucleic acid molecule comprises a ribonucleic acid molecule.

17. A method as recited in claim 10, wherein said nucleic acid molecule comprises a peptide nucleic acid molecule.

18. A method as recited in claim 10, wherein said nucleic acid molecule comprises a single strand of a deoxyribonucleic acid molecule hybridized to a single strand of a ribonucleic acid molecule.

19. A method as recited in claim 10, wherein said step of bonding comprises the steps of:
(a) attaching said phosphoramidite-containing molecule to a first end of an oligonucleotide, thereby forming an altered oligonucleotide;
(b) hybridizing said altered oligonucleotide to said first end of said nucleic acid molecule; and
(c) extending said altered oligonucleotide to a length substantially the same as a length of said nucleic acid molecule.

20. A method for producing a fusion molecule capable of use as a detector molecule for binding a predetermined target analyte, said method comprising the steps of:
(a) attaching a reactive moiety to a first end of a protein sub-unit, thereby creating a reactive intermediate with said reactive moiety at a first end thereof;
(b) attaching a coupling reagent to a nucleotide, thereby forming a modified nucleotide;
(c) linking said modified nucleotide to a first end of a nucleic acid molecule, thereby forming a modified nucleic acid molecule, said coupling reagent of said modified nucleic acid molecule being capable of displacing said reactive moiety of said reactive intermediate; and
(d) catalyzing a reaction between said reactive moiety of said reactive intermediate and said coupling reagent of said modified nucleic acid molecule, wherein in said reaction:
(i) said reactive moiety is displaced from said first end of said reactive intermediate; and
(ii) a covalent bond is formed between a first end of said reactive intermediate and said first end of said modified nucleic acid molecule.

21. A method as recited in claim 20, wherein said nucleic acid molecule comprises a deoxyribonucleic acid molecule.

22. A method as recited in claim 21, wherein said deoxyribonucleic acid molecule comprises a double-stranded deoxyribonucleic acid molecule.

23. A method as recited in claim 22, wherein said step of linking comprises the steps of:
(a) removing a terminal nucleotide from a first end of said double-stranded deoxyribonucleic acid molecule; and
(b) replacing said terminal nucleotide removed from said double-stranded deoxyribonucleic acid molecule with said modified nucleotide.

24. A method as recited in claim 20, wherein said coupling reagent comprises a cysteine-like moiety.

25. A method as recited in claim 20, wherein said reactive moiety comprises a thiol-containing group.

26. A method as recited in claim 20, wherein said nucleotide comprises 2′-aminoallele-deoxyuracil tri-phosphate.

27. A method as recited in claim 20, wherein said modified nucleotide comprises cysteine modified deoxy-uracil tri-phosphate.

28. A method for producing a fusion molecule capable of use as a detector molecule for binding a predetermined target analyte, said method comprising the steps of:
(a) attaching a reactive moiety to a first end of a protein sub-unit, thereby creating a reactive intermediate with said reactive moiety at a first end thereof;
(b) attaching a cysteine-like moiety to a nucleotide, thereby forming a modified nucleotide;
(c) linking a first end of a nucleic acid molecule to said modified nucleotide, thereby forming a modified nucleic acid molecule, said cysteine-like moiety of said modified nucleic acid molecule being capable of displacing said reactive moiety of said reactive intermediate; and
(d) catalyzing a reaction between said reactive moiety of said reactive intermediate and said cysteine-like moiety of said modified nucleic acid molecule, wherein in said reaction:
(i) said reactive moiety is displaced from said first end of said reactive intermediate; and
(ii) a covalent bond is formed between said first end of said reactive intermediate and said first end of said modified nucleic acid molecule.

29. A method as recited in claim 28, further comprising the steps of:
(a) determining a first nucleic acid sequence encoding for said protein sub-unit;
(b) cloning said first nucleic acid sequence into a vector having a second nucleic acid sequence encoding for an intein segment, thereby producing in said vector a protein sub-unit-intein fusion sequence;
(c) expressing said protein sub-unit-intein fusion sequence in a cell, thereby producing a protein sub-unit-intein recombinant protein; and
(d) adding said reactive moiety and tris-(2-carboxyethyl) phosphine to a solution containing said protein sub-unit-intein recombinant protein, thereby inducing a hydrolysis reaction wherein:
(i) the intein portion of said protein sub-unit-intein recombinant protein is removed therefrom; and
(ii) said reactive moiety becomes attached to said first end of said protein sub-unit.

30. A method as recited in claim 28, wherein said cysteine-like moiety comprises cysteine.

31. A method as recited in claim 28, wherein said nucleotide comprises 2′-aminoallele-deoxyuracil tri-phosphate.

32. A method as recited in claim 31, wherein said modified nucleotide comprises cysteine modified deoxy-uracil tri-phosphate.

33. A method as recited in claim 28, wherein said reactive moiety comprises a thiol-containing group.

34. A method as recited in claim 33, wherein said thiol-containing group is selected from the group consisting of thiophenol and mercaptoethanesulfonic acid.

35. A method as recited in claim 28, wherein said covalent bond comprises an amide bond.

36. A method for producing a fusion molecule capable of use as a detector molecule for binding a predetermined target analyte, said method comprising the steps of:
(a) attaching reactive moieties to first ends of protein sub-units of a quantity of protein sub-units, thereby creating a quantity of reactive intermediates with said reactive moieties at first ends thereof;
(b) bonding first coupling reagents to first nucleotides, thereby forming first modified nucleotides;
(c) connecting second coupling reagents to second nucleotides, thereby forming second modified nucleotides;
(d) linking first ends of a quantity of nucleic acid molecules to said first modified nucleotides, and linking second ends of said quantity of said nucleic acid molecules to said second modified nucleotides, thereby forming modified nucleic acid molecules with first and second ends;
(e) severing said modified nucleic acid molecules between said first and said second ends of said nucleic acid thereof, thereby forming:
(i) from said first end from said modified nucleic acid first modified nucleic acid fragments containing said first modified nucleotide; and
(ii) from said second end from said modified nucleic acid second modified nucleic acid fragments containing said second modified nucleotide; and
(f) catalyzing a first reaction between said first coupling reagent of said first modified nucleic acid fragments and reactive moieties of said reactive intermediates of said quantity thereof, wherein in said reaction:
(i) said reactive moieties are displaced from said first ends of said reactive intermediates; and
(ii) first covalent bonds are formed between said reactive intermediate and said first modified nucleotide of said first modified nucleic acid fragment;
(g) catalyzing a second reaction between said second coupling reagent of said second modified nucleic acid fragments and reactive moieties of said reactive intermediates of said quantity thereof, wherein in said reaction:
(i) said reactive moieties are displaced from said reactive intermediates; and
(ii) second covalent bonds are formed between said reactive intermediates and said second modified nucleotides of said second modified nucleic acid fragments.

37. A method as recited in claim 36, wherein said first nucleotides are substantially identical to said second nucleotides.

38. A method as recited in claim 36, wherein said step of severing produces modified nucleic acid fragments of substantially equal length.

39. A method as recited in claim 36, wherein said step of severing comprises digesting said modified nucleic acid molecules with a first restriction enzyme.

40. A method as recited in claim 36, wherein said first covalent bonds are substantially the same as said second covalent bonds.

41. A method as recited in claim 36, wherein said first and second covalent bonds comprise amide bonds.

42. A method as recited in claim 36, wherein said first modified nucleotides are substantially identical to said second modified nucleotides.

43. A method as recited in claim 36, wherein said first modified nucleotides and said second modified nucleotides comprise cysteine modified deoxy-uracil tri-phosphate.

44. A method as recited in claim 36, wherein said first restriction enzyme comprises EcoRI.

45. A method as recited in claim 36, wherein said step of linking comprises the steps of:
(a) digesting said quantity of nucleic acid molecules with a second restriction enzyme, thereby producing nucleotide overhangs at said first and second ends; and
(b) filling in said nucleotide overhangs at said first and second ends of said quantity of nucleic acid molecules using said first and second modified nucleotides.

46. A fusion molecule capable of binding a predetermined target analyte, said fusion molecule comprising:
(a) a protein sub-unit;
(b) a linker attached to a first end of said protein sub-unit; and
(c) a deoxyribonucleic acid molecule attached at a first end thereof to said linker by a covalent bond.

47. A fusion molecule as recited in claim 46, wherein said linker comprises an amide linkage.

48. A fusion molecule as recited in claim 46, wherein said deoxyribonucleic acid molecule comprises a double-stranded deoxyribonucleic acid molecule.

49. A fusion molecule as recited in claim 46, wherein said covalent bond comprises an amide bond.

50. A fusion molecule as recited in claim 46, wherein said linker comprises a cysteine-like moiety.

51. A fusion molecule as recited in claim 46, wherein said first end of said protein sub-unit comprises a carboxyl terminus of said protein sub-unit.

52. A fusion molecule as recited in claim 46, wherein said first end of said deoxyribonucleic acid molecule comprises the 5′ terminus of said deoxyribonucleic acid molecule.

53. A fusion molecule as recited in claim 46, wherein said first end of said deoxyribonucleic acid molecule comprises the 3′ terminus of said deoxyribonucleic acid molecule.

54. A fusion molecule as recited in claim 46, wherein said deoxyribonucleic acid molecule is a barcode identifying said protein sub-unit.

55. A fusion molecule capable of binding a predetermined target analyte, said fusion molecule comprising:
(a) a protein sub-unit;
(b) a cysteine-like moiety attached to a first end of said protein sub-unit; and
(c) a nucleic acid molecule attached at a first end thereof to said cysteine-like moiety by a covalent bond.

56. A fusion molecule as recited in claim 55, wherein said nucleic acid molecule comprises a deoxyribonucleic acid molecule.

57. A fusion molecule as recited in claim 55, wherein said nucleic acid molecule comprises a ribonucleic acid molecule.

58. A fusion molecule as recited in claim 55, wherein said first end of said protein-subunit comprises a carboxyl terminus.

59. A fusion molecule as recited in claim 55, wherein said first end of said nucleic acid molecule comprises the 5′ end of said nucleic acid molecule.

60. A fusion molecule as recited in claim 55, wherein said cysteine-like moiety comprises an amide linkage.

61. A fusion molecule as recited in claim 56, wherein said deoxyribonucleic acid molecule comprises a double-stranded deoxyribonucleic acid molecule.

62. A fusion molecule as recited in claim 55, wherein said covalent bond comprises an amide bond.

63. A fusion molecule as recited in claim 55, wherein said nucleic acid molecule comprises a peptide nucleic acid molecule.

64. A fusion molecule as recited in claim 55, wherein said first end of said nucleic acid molecule comprises the 3′ end of said nucleic acid molecule.

65. The product of a process comprising:
(a) attaching a reactive moiety to a first end of a protein sub-unit, thereby creating a reactive intermediate with said reactive moiety at a first end thereof;
(b) bonding a coupling reagent to a first end of a nucleic acid molecule, thereby forming a modified nucleic acid molecule, said coupling reagent of said modified nucleic acid molecule being capable of displacing said reactive moiety of said reactive intermediate; and
(c) catalyzing a reaction between said reactive moiety of said reactive intermediate and said coupling reagent of said modified nucleic acid molecule, wherein in said reaction:
(i) said reactive moiety is displaced from said first end of said reactive intermediate; and
(ii) a covalent bond is formed between said first end of said reactive intermediate and said first end of said modified nucleic acid molecule.

66. A method as recited in claim 65, wherein said first end of said protein sub-unit comprises a carboxyl terminus of said protein sub-unit.

67. The product as recited in claim 65, wherein said reactive moiety comprises a thiol-containing group.

68. The product as recited in claim 65, wherein said bonding step comprises the steps of:
(a) attaching said coupling reagent to a nucleotide, thereby forming a modified nucleotide; and
(b) linking a first end of said nucleic acid molecule to said modified nucleotide to form a modified nucleic acid molecule.

69. The product as recited in claim 65, wherein said coupling reagent comprises a cysteine-like moiety.

70. The product as recited in claim 65, wherein said covalent bond comprises an amide bond.

71. The product as recited in claim 65, wherein said protein sub-unit comprises a natural protein.

72. The product as recited in claim 65, wherein said protein sub-unit comprises a recombinant peptide aptamer.

73. The product as recited in claim 65, wherein said protein sub-unit comprises a synthetic peptide.

74. The product as recited in claim 67, wherein said thiol-containing group is selected from the group consisting of thiophenol and mercaptoethanesulfonic acid.

75. The product of a process comprising:
(a) attaching a reactive moiety to a first end of a protein sub-unit, thereby creating a reactive intermediate with said reactive moiety attached at a first end thereof;
(b) bonding first coupling reagents to first nucleotides, thereby forming first modified nucleotides;
(c) connecting second coupling reagents to second nucleotides, thereby forming second modified nucleotides;
(d) linking first ends of a quantity of nucleic acid molecules to said first modified nucleotides, and linking second ends of said quantity of said nucleic acid molecules to said second modified nucleotides, thereby forming modified nucleic acid molecules with first and second ends;
(e) severing said modified nucleic acid molecules between said first and said second ends of said nucleic acid molecules thereof, thereby forming:
(i) from said first end from said modified nucleic acid molecule first modified nucleic acid fragments containing said first modified nucleotide; and
(ii) from said second end from said modified nucleic acid molecule second modified nucleic acid fragments containing said second modified nucleotide; and
(f) catalyzing a first reaction between said first coupling reagent of said first modified nucleic acid fragments and reactive moieties of said reactive intermediates of said quantity thereof, wherein in said reaction:
(i) said reactive moiety is displaced from said first end of said reactive intermediate; and
(ii) a first covalent bond is formed between said reactive intermediate and said first modified nucleotide of said first modified nucleic acid fragment;
(g) catalyzing a second reaction between said second coupling reagent of said second modified nucleic acid fragments and reactive moieties of said reactive intermediates of said quantity thereof, wherein in said reaction:
(i) said reactive moieties are displaced from said reactive intermediates; and
(ii) second covalent bonds are formed between said reactive intermediates and said second modified nucleotides of said second modified nucleic acid fragments.

76. The product as recited in claim 75, wherein said first modified nucleotides are substantially identical to said second modified nucleotides.

77. The product as recited in claim 75, wherein said step of severing produces modified nucleic acid fragments of substantially equal length.

78. The product as recited in claim 75, wherein said reactive moiety comprises a thiol-containing group.

79. The product as recited in claim 75, wherein said first and second coupling reagents comprise cysteine-like moieties.

80. The product as recited in claim 75, wherein said step of severing comprises digesting said modified nucleic acid molecules with a restriction enzyme.

81. The product as recited in claim 75, wherein said first modified nucleotides and said second modified nucleotides comprise cysteine modified deoxy-uracil tri-phosphate.

82. The product as recited in claim 75, wherein said quantity of modified nucleic acids comprise a T7 promoter sequence.

83. A method for recognizing a target analyte in a sample, said method comprising the steps of:
(a) manufacturing a quantity of detector fusion molecules, said step of manufacturing comprising the steps of:
(i) attaching a quantity of reactive moieties to a first end of a quantity of protein sub-units capable of binding to the target analyte, thereby creating a quantity of reactive intermediates with said reactive moieties at first ends thereof;
(ii) bonding a quantity of coupling reagents to first ends of a quantity of nucleic acid molecules, thereby forming a quantity of modified nucleic acid molecules, said coupling reagent of said modified nucleic acid molecules being capable of displacing said reactive moiety of said reactive intermediates; and
(iii) catalyzing a reaction between said reactive moiety of said reactive intermediates and said coupling reagent of said modified nucleic acid molecules, wherein in said reaction:
(A) said reactive moiety is displaced from said first ends of said reactive intermediates; and
(B) a covalent bond is formed between said first end of said reactive intermediates and said first end of said modified nucleic acid molecules;
(b) contacting the sample with said quantity of said detector fusion molecules, whereby said detector fusion molecules from said quantity thereof bind to the target analyte in the sample;
(c) amplifying said nucleic acid molecules of said detector fusion molecules bound to the target analyte, thereby producing an amplification product; and
(d) identifying said amplification product.

84. A method as recited in claim 83, further comprising the steps of:
(a) immobilizing said sample on a substrate; and
(b) washing from said sample immobilized on said substrate said detector fusion molecules unbound to said sample.

85. A method as recited in claim 83, wherein said step of amplifying comprises the steps of:
(a) hybridizing a primer having a nucleotide sequence complementary to a portion of a sequence of said modified nucleic acid molecules to said modified nucleic acid molecule of said bound detector fusion molecule; and
(b) adding a deoxyribonucleic acid polymerase to said bound detector fusion molecule.

86. A method as recited in claim 83, wherein said step of amplifying comprises adding a ribonucleic acid polymerase to said bound detector fusion molecule.

87. A method as recited in claim 83, wherein said step of identifying comprises the steps of:
(a) resolving said amplification product on a basis of size; and
(b) staining said amplification product.

88. A method as recited in claim 83, wherein said covalent bond comprises an amide bond.

89. A method as recited in claim 85, wherein said primer further comprises a detectable marker.

90. A method as recited in claim 89, wherein said step of identifying comprises sensing said detectable marker.

91. A method as recited in claim 83, wherein said step of identifying comprises determining a sequence of said amplification product.

92. A method as recited in claim 83, wherein said nucleic acid molecules in said quantity of nucleic acid molecules vary in length.

93. A method for quantifying a target analyte in a sample, said method comprising the steps of:
(a) manufacturing a quantity of detector fusion molecules, said step of manufacturing comprising:
(i) attaching a quantity of reactive moieties to a first end of a quantity of protein sub-units capable of binding to the target analyte, thereby creating a quantity of a reactive intermediates with said reactive moieties at first ends thereof;
(ii) bonding a quantity of coupling reagents to a first ends of a quantity of nucleic acid molecules, thereby forming a quantity of modified nucleic acid molecules, said coupling reagent of said modified nucleic acid molecule being capable of displacing said reactive moiety of said reactive intermediates; and
(iii) catalyzing a reaction between said reactive moiety of said reactive intermediates and said coupling reagent of said modified nucleic acid molecules, wherein of said reaction:
(A) said reactive moiety is displaced from said first ends of said reactive intermediates; and
(B) a covalent bond is formed between said first end of said reactive intermediates and said first end of said modified nucleic acid molecules;
(b) contacting the sample with said quantity of said detector fusion molecules, whereby said detector fusion molecules from said quantity thereof bind to the target analyte in the sample;
(c) amplifying said nucleic acid molecules of said quantity of said detector fusion molecules bound to the target analyte, thereby producing an amplification product; and
(d) determining an amount of said amplification product.

94. A method as recited in claim 93, wherein said step of amplifying comprises the steps of:
(a) hybridizing a primer having a nucleotide sequence complementary to a sequence of said modified nucleic acid molecule to said modified nucleic acid molecules of said bound detector fusion molecule; and
(b) adding a deoxyribonucleic acid polymerase to said bound detector fusion molecule.

95. A method as recited in claim 93, wherein said step of amplifying comprises adding a ribonucleic acid polymerase to said bound detector fusion molecule.

96. A method as recited in claim 93, wherein said step of determining comprises the steps of:
(a) attaching a probe to a microarray chip;
(b) querying said probe on said microarray chip with said amplification product; and
(c) determining a number of said amplification products that hybridize to said probe of said microarray chip.

97. A method as recited in claim 94, wherein said primer further comprises a detectable marker.

98. A method as recited in claim 97, wherein said step of determining comprises measuring an amount of said detectable marker.

99. A method as recited in claim 93, further comprising resolving said amplification product on a basis of size.

100. A method as recited in claim 99, wherein said step of determining comprises measuring an amount of said amplification product resolved on said gel.

101. A method as recited in claim 93, wherein said protein sub-units in said quantity of protein sub-units recognize different target analytes.

102. A method as recited in claim 98, wherein said detectable marker is an intercalating fluorescent dye.

103. A method for creating a nanostructure on a target analyte using a detector fusion molecule, said method comprising the steps of:
(a) manufacturing a quantity of detector fusion molecules, said step of manufacturing comprising:
(i) attaching a quantity of reactive moieties to a first end of a quantity of protein sub-units capable of binding to the target analyte, thereby creating a quantity of a reactive intermediates with said reactive moieties at first ends thereof;
(ii) bonding a quantity of coupling reagents to a first ends of a quantity of nucleic acid molecules, thereby forming a quantity of modified nucleic acid molecules, said coupling reagent of said modified nucleic acid molecule being capable of displacing said reactive moiety of said reactive intermediates; and
(iii) catalyzing a reaction between said reactive moiety of said reactive intermediates and said coupling reagent of said modified nucleic acid molecules, wherein of said reaction:
(A) said reactive moiety is displaced from said first ends of said reactive intermediates; and
(B) a covalent bond is formed between said first end of said reactive intermediates and said first end of said modified nucleic acid molecules;
(b) attaching a target analyte to a substrate, thereby forming a multimeric complex;
(c) binding said detector fusion molecule to said multimeric complex, thereby forming a fusion molecule-analyte complex; and
(d) linking a higher order structure to said fusion molecule-analyte complex.

104. A method as recited in claim 103, wherein said attaching step comprises attaching a plurality of said target analytes to said substrate, thereby forming a plurality of said multimeric complexes.

105. A method as recited in claim 104, wherein said binding step comprises binding a plurality of detector fusion molecules to said plurality of said fusion-molecule-analyte complexes, thereby forming a plurality of said fusion molecule-analyte complexes.

106. A method as recited in claim 105, wherein said linking step further comprises linking a plurality of said higher order structures to said plurality of said fusion molecule-analyte complexes.

107. A method as recited in claim 103, wherein said higher order structure comprises a polypeptide.

108. A kit for use in recognizing or quantifying a target analyte, said kit comprising:
(a) a detector fusion molecule capable of binding to a target analyte, said detector fusion molecule comprising:
(i) a protein sub-unit;
(ii) a linker attached to a first end of said protein sub-unit; and
(iii) a deoxyribonucleic molecule attached at a first end thereof to said linker by a covalent bond;
(b) first means for amplifying said detector fusion molecule, thereby producing an amplification product; and
(c) second means for visualizing said amplification product.

109. A kit as recited in claim 108, wherein said first means comprises a ribonucleic acid polymerase.

110. A kit as recited in claim 109, wherein said ribonucleic acid polymerase comprises a T7 ribonucleic acid polymerase.

111. A kit as recited in claim 110, wherein said deoxyribonucleic acid molecule includes a T7 promoter sequence.

112. A kit as recited in claim 109, wherein said first means further comprises a ribonucleic acid primer.

113. A kit as recited in claim 108, wherein said first means comprises:
(a) a deoxyribonucleic acid primer; and
(b) a deoxyribonucleic acid polymerase.

114. A kit as recited in claim 113, wherein said deoxyribonucleic acid polymerase is selected from the group consisting of Klenow, Tax polymerase, Vent polymerase, and Deep Vent polymerase.

115. A kit as recited in claim 113, wherein said second means comprises a detectable marker attached to said deoxyribonucleic acid primer.

116. A kit as recited in claim 108, wherein said linker comprises a cysteine-like moiety.

117. A kit as recited in claim 108, wherein said covalent bond comprises an amide bond.
